SEQUENCE LISTING (5 sequences)

SEQ ID NO: 1 - DNA, 57 bp, Artificial sequence
  Synthetic shRNA targeting human Notch1
ccggctttgtttcaggttcagtattctcgagaatactgaacctgaaacaaagttttt

SEQ ID NO: 2 - DNA, 57 bp, Artificial sequence
  Synthetic shRNA targeting human Notch1
ccggcgctgcctggacaagatcaatctcgagattgatcttgtccaggcagcgttttt

SEQ ID NO: 3 - DNA, 57 bp, Artificial sequence
  Synthetic shRNA targeting human Notch1
ccggcaaagacatgaccagtggctactcgagtagccactggtcatgtctttgttttt

SEQ ID NO: 4 - DNA, 5820 bp, Homo sapiens
agccgctgcgcccgagctggcctgcgagttcagggctcctgtcgctctccaggagcaacctctactccggacgcacaggcattccccgcgcccctccagccctcgccgccctcgccaccgctcccggccgccgcgctccggtacacacaggatccctgctgggcaccaacagctccaccatggggctggcctggggactaggcgtcctgttcctgatgcatgtgtgtggcaccaaccgcattccagagtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgggcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccagggggtgctgcagaatgtgaggtttgtctttggaaccacaccagaagacatcctcaggaacaaaggctgctccagctctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtgactgaagagaacaaagagttggccaatgagctgaggcggcctcccctatgctatcacaacggagttcagtacagaaataacgaggaatggactgttgatagctgcactgagtgtcactgtcagaactcagttaccatctgcaaaaaggtgtcctgccccatcatgccctgctccaatgccacagttcctgatggagaatgctgtcctcgctgttggcccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaagagatttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgccccatcaatggaggctggggtccttggtcaccatgggacatctgttctgtcacctgtggaggaggggtacagaaacgtagtcgtctctgcaacaaccccacaccccagtttggaggcaaggactgcgttggtgatgtaacagaaaaccagatctgcaacaagcaggactgtccaattgatggatgcctgtccaatccctgctttgccggcgtgaagtgtactagctaccctgatggcagctggaaatgtggtgcttgtccccctggttacagtggaaatggcatccagtgcacagatgttgatgagtgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacaggtgtgcaagccccgtaacccctgcacggatgggacccacgactgcaacaagaacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaaggataattgccccaaccttcccaactcagggcaggaagactatgacaaggatggaattggtgatgcctgtgatgatgacgatgacaatgataaaattccagatgacagggacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacggtatcctcaatgaacgggacaactgccagtacgtctacaatgtggaccagagagacactgatatggatggggttggagatcagtgtgacaattgccccttggaacacaatccggatcagctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacggcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatcccaaaatgaccctaactgggttgtacgccatcagggtaaagaactcgtccagactgtcaactgtgatcctggactcgctgtaggttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacaggaaacacccctggccaggtgcgcaccctgtggcatgaccctcgtcacataggctggaaagatttcaccgcctacagatggcgtctcagccacaggccaaagacgggtttcattagagtggtgatgtatgaagggaagaaaatcatggctgactcaggacccatctatgataaaacctatgctggtggtagactagggttgtttgtcttctctcaagaaatggtgttcttctctgacctgaaatacgaatgtagagatccctaatcatcaaattgttgattgaaagactgatcataaaccaatgctggtattgcaccttctggaactatgggcttgagaaaacccccaggatcacttctccttggcttccttcttttctgtgcttgcatcagtgtggactcctagaacgtgcgacctgcctcaagaaaatgcagttttcaaaaacagactcagcattcagcctccaatgaataagacatcttccaagcatataaacaattgctttggtttccttttgaaaaagcatctacttgcttcagttgggaaggtgcccattccactctgcctttgtcacagagcagggtgctattgtgaggccatctctgagcagtggactcaaaagcattttcaggcatgtcagagaagggaggactcactagaattagcaaacaaaaccaccctgacatcctccttcaggaacacggggagcagaggccaaagcactaaggggagggcgcatacccgagacgattgtatgaagaaaatatggaggaactgttacatgttcggtactaagtcattttcaggggattgaaagactattgctggatttcatgatgctgactggcgttagctgattaacccatgtaaataggcacttaaatagaagcaggaaagggagacaaagactggcttctggacttcctccctgatccccacccttactcatcacctgcagtggccagaattagggaatcagaatcaaaccagtgtaaggcagtgctggctgccattgcctggtcacattgaaattggtggcttcattctagatgtagcttgtgcagatgtagcaggaaaataggaaaacctaccatctcagtgagcaccagctgcctcccaaaggaggggcagccgtgcttatatttttatggttacaatggcacaaaattattatcaacctaactaaaacattccttttctcttttttcctgaattatcatggagttttctaattctctcttttggaatgtagattttttttaaatgctttacgatgtaaaatatttattttttacttattctggaagatctggctgaaggattattcatggaacaggaagaagcgtaaagactatccatgtcatctttgttgagagtcttcgtgactgtaagattgtaaatacagattatttattaactctgttctgcctggaaatttaggcttcatacggaaagtgtttgagagcaagtagttgacatttatcagcaaatctcttgcaagaacagcacaaggaaaatcagtctaataagctgctctgccccttgtgctcagagtggatgttatgggattctttttttctctgttttatcttttcaagtggaattagttggttatccatttgcaaatgttttaaattgcaaagaaagccatgaggtcttcaatactgttttaccccatcccttgtgcatatttccagggagaaggaaagcatatacacttttttctttcatttttccaaaagagaaaaaaatgacaaaaggtgaaacttacatacaaatattacctcatttgttgtgtgactgagtaaagaatttttggatcaagcggaaagagtttaagtgtctaacaaacttaaagctactgtagtacctaaaaagtcagtgttgtacatagcataaaaactctgcagagaagtattcccaataaggaaatagcattgaaatgttaaatacaatttctgaaagttatgttttttttctatcatctggtataccattgctttatttttataaattattttctcattgccattggaatagatatctcagattgtgtagatatgctatttaaataatttatcaggaaatactgcctgtagagttagtatttctatttttatataatgtttgcacactgaattgaagaattgttggttttttcttttttttgttttgtttttttttttttttttttttgcttttgacctcccatttttactatttgccaatacctttttctaggaatgtgcttttttttgtacacatttttatccattttacattctaaagcagtgtaagttgtatattactgtttcttatgtacaaggaacaacaataaatcatatggaaatttatatttataaaaaaaaaaaaaaaaaaaaaaaaaaaaaaa

SEQ ID NO: 5 - protein, 1170 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

(19) United States
(12) Patent Application Publication
(10) Pub. No.: US 2017/0114329 A1
(43) Pub. Date: Apr. 27, 2017

(54) SCALABLE ORGANOTYPIC MODELS OF TUMOR DORMANCY

(71) Applicants: Cyrus M. Ghajar, Greenbrae, CA (US); Mina J. Bissell, Berkeley, CA (US)

(72) Inventors: Cyrus M. Ghajar, Greenbrae, CA (US); Mina J. Bissell, Berkeley, CA (US)

(73) Assignee: THE REGENTS OF THE UNIVERSITY OF CALIFORNIA, Oakland, CA (US)

(21) Appl. No.: 14/944,137

(22) Filed: Nov. 17, 2015

Related U.S. Application Data
(63) Continuation of application No. PCT/US2014/038514, filed on May 17, 2014.
(60) Provisional application No. 61/824,949, filed on May 17, 2013.

(51) Int. Cl. C12N 5/071; G01N 33/50

(52) U.S. Cl. CPC C12N 5/0697; G01N 33/5011; G01N 33/5064; G01N 33/5044; C12N 2502/28; C12N 2502/13

(57) ABSTRACT

Herein are described synthetic organotypic microvascular niches formed by self-assembly of stromal cells cultured endothelial cells seeded with cells of interest to model and determine dormancy state of these cells of interest in these tissues. These models demonstrated that endothelial-derived thrombospondin-1 induces sustained cancer cell quiescence. We further describe dormancy models, and identified active tumor-promoting, endothelial tip cell-derived factors. Our work reveals that stable microvasculature constitutes a ‘dormant niche,’ whereas sprouting neovasculature sparks micrometastatic outgrowth.

### CROSS-REFERENCE TO RELATED APPLICATIONS

This application is a continuation of and claims priority to International Patent Application No. PCT/US2014/038514, filed on May 17, 2014, and U.S. Provisional Patent Application No. 61/824,949, filed on May 17, 2013, which are hereby incorporated by reference in their entirety.

### STATEMENT OF GOVERNMENTAL SUPPORT

The present invention was supported by Contract No. DE-AC02-05CH11231 awarded by the U.S. Department of Energy and by Grant Nos. CA126552 and CA143836 awarded by the National Institutes of Health. The government has certain rights to the invention.

### REFERENCE TO SEQUENCE LISTING APPENDIX

A sequence listing is submitted concurrently with the specification and is part of the specification and is hereby incorporated in its entirety by reference herein. This application also incorporates by reference the sequence listing found in computer-readable form in a *.txt file entitled, “2013-097-03US_SequenceListing_ST25.txt”, created on Nov. 17, 2015.

### FIELD OF THE INVENTION

The present invention relates to systems and tissue models for cancer research and methods for screening for biomarkers. The present invention also relates to tumor or cancer suppressor proteins and biomarkers.

### BACKGROUND OF THE INVENTION

It has been difficult if not impossible to predict if and when metastases will occur (See Aguirre-Ghiso, J. A. Models, mechanisms and clinical evidence for cancer dormancy. Nat Rev Cancer 7, 834-846 (2007)). The reason is that although the metastatic cascade is depicted typically as a linear process, in reality it is anything but. Some patients may experience metastatic relapse within months whereas others go several years or even decades without distant recurrence (See Aguirre-Ghiso, J. A. Models, mechanisms and clinical evidence for cancer dormancy. Nat Rev Cancer 7, 834-846 (2007); Goss, P. E. & Chambers, A. F. Does tumour dormancy offer a therapeutic target? Nat Rev Cancer 10, 871-877 (2010); Klein, C. A. Parallel progression of primary tumours and metastases. Nat Rev Cancer 9, 302-312 (2009); Uhr, J. W. & Pantel, K. Controversies in clinical cancer dormancy. Proc Natl Acad Sci USA 108, 12396-12400 (2011)). The recent discovery of tumor promoting milieus (referred to as metastatic niches (See Kaplan, R. N., et al. VEGFR1-positive haematopoietic bone marrow progenitors initiate the pre-metastatic niche. Nature 438, 820-827 (2005); Peinado, H., et al. Melanoma exosomes educate bone marrow progenitor cells toward a pro-metastatic phenotype through MET. Nat Med 18, 883-891 (2012); Psaila, B. & Lyden, D. The metastatic niche: adapting the foreign soil. Nat Rev Cancer 9, 285-293 (2009)) established at distant sites prior to- or upon-the arrival of disseminated tumor cells (DTCs) could explain the population that relapses early. But in late relapsing populations, what tumor cells do from the time of dissemination to the time they become clinically detectable is an outstanding question. Studies in mice and analysis of human clinical specimens revealed that single- or small clusters of DTCs may persist long-term in a state of quiescence (Suzuki, M., Mose, E. S., Montel, V. & Tarin, D. Dormant cancer cells retrieved from metastasis-free organs regain tumorigenic and metastatic potency. Am J Pathol 169, 673-681 (2006); Pantel, K., et al. Differential expression of proliferation-associated molecules in individual micrometastatic carcinoma cells. J Natl Cancer Inst 85, 1419-1424 (1993)). Precisely where these cells reside, how they are induced into a dormant state and what eventually causes them to ‘awaken’ remain perplexing mysteries in tumor biology. Solving these problems is key to designing therapies that prevent relapse by either sustaining tumor dormancy or by selectively killing off dormant cells with minimal damage to normal tissues¹¹.

Dealing with DTCs while they are dormant is desirable because one could then pre-empt metastatic disease. However, these cells persist despite the application of targeted- and chemo-therapies. Essentially, there are two options to prevent dormant DTCs from becoming a problem: 1) Maintain them in a state of dormancy indefinitely; or 2) disrupt the interactions between dormant DTCs and their microenvironment (the healthy tissue that surrounds them) in order to render them susceptible to subsequently applied chemotherapeutics. Doing either requires that we understand how these cells are steered into and maintained in a state of dormancy in the first place.

We have long argued and provided evidence that basement membrane (BM), in particular laminin-111, provides a hospitable microenvironment that allows mammary epithelial cell survival, quiescence and resistance to cytotoxic agents, three properties commonly associated also with dormant DTCs (Braun, S., et al. A pooled analysis of bone marrow micrometastasis in breast cancer. N Engl J Med 353, 793-802 (2005)). Thus, we suspected that BM was a major component of the ‘dormant niche’ in distant organs. Given that breast cancer cells (BCCs) must take a haematogenous route to arrive at sites where breast tumors metastasize most often (i.e., lung, bone marrow (BoMa), brain and liver)(Chambers, A. F., Groom, A. C. & MacDonald, I. C. Dissemination and growth of cancer cells in metastatic sites. Nat Rev Cancer 2, 563-572 (2002)), the microvascular BM would be the first of its kind encountered by tumor cells as they disseminate to these tissues. Therefore, we reasoned that endothelial cells (ECs)—and factors deposited within their surrounding BM—may be a prime player within the dormant niche.

To date, no study has demonstrated a means to naturally steer full-blown tumor cells into a state of dormancy without using exogenous inhibitors.

Accordingly, there is a need for effective models and methods for modeling microenvironments that promote or suppress dormancy of tumor cells.

### SUMMARY OF THE INVENTION

In some embodiments, the present model allows the in vitro organotypic modeling of microvascular niches from various tissues. In various embodiments, the present models comprised of a specific stromal cell type of the endothelial cells, and either endothelial cells from the particular tissue or human umbilical vein endothelial cells (HUVEC).

As shown in the Examples, we first localized dormant DTCs from the breast to the microvasculature of the lung, bone marrow and brain, and then constructed organotypic models of lung- and bone marrow-microvascular niches—that are maintained in serum- and cytokine-free conditions—in order to demonstrate that endothelium directly regulates tumor dormancy. We have discovered that stable endothelium induces tumor quiescence whereas disruption of stable endothelium accelerates tumor outgrowth.

In various embodiments, the present invention provides for models that may be used to provide the microvascular niches that model the most common tissue sites of relapse in cancer where slow-growing or dormant tumor cells may be found. In some embodiments, stromal cells and endothelial cells are combined, allowed to self-assemble and form complexes that model microvascular niches.

In some embodiments, a tissue model for in vitro organotypic modeling of dormancy in a microvascular niche comprising: (a) stromal cells of a selected specific stromal cell type from a particular tissue; (b) endothelial cells, wherein the endothelial cells are from the particular tissue or human umbilical vein endothelial cells (HUVEC), wherein the stromal cells and the endothelial cells self-assembled to form a microvascular niche, and (c) seeded cells of interest. In some embodiments, the tissue model can further comprise other seeded resident cells, wherein the resident cells are cells that reside in vivo in the particular tissue being modeled. In other embodiments, the tissue model can further comprise seeded non-resident cells, wherein the non-resident cells are cells that do not reside in or are generated in vivo from the particular tissue being modeled. In various embodiments, the tissue that is modeled is lung, brain, bone marrow, liver, lymph node, ovary, omentum, pancreas, skeletal muscle, heart, skin, breast, prostate, kidney, or bladder.

A method for forming a synthetic organotypic model of dormancy in a microvascular niche comprising the steps of (a) contacting stromal cells with endothelial cells, wherein said stromal cells are of a specific cell type from the tissue being modeled, (b) allowing the stromal cells and endothelial cells to self-assemble and form three-dimensional (3D) complexes that model microvascular niches; and (c) culturing or seeding cells of interest in the 3D complexes. In some embodiments, the method, further comprising the step of (d) detecting dormancy or growth of said seeded cells.

Thus in one embodiment, to form a lung tissue microvascular niche, lung fibroblasts and HUVEC or lung endothelial cells can be used. In another embodiment, to form a bone marrow microvascular niche, mesenchymal stem cells and HUVEC or bone marrow endothelial cells may be used. In another embodiment, to form a brain microvascular niche, human adventitial fibroblasts and astrocytes, and HUVEC or endothelial cells may be used. In yet another embodiment, to form a liver microvascular niche, liver stellate cells and endothelial cells or HUVEC can be used.

In various embodiments, the present engineered models may be used as high-throughput screening tools and in conjunction with—OMICS technologies (e.g., proteomics) in order to identify factors that characterize the dormant niche, induce tumor cells into a state of dormancy or draw them out of this state.

In some embodiments, the endothelial cells are human umbilical vein endothelial cells (HUVEC). In one embodiment, the HUVEC can be transduced with a lentiviral construct containing the human adenoviral E4ORF1 gene. In another embodiment, endothelial cells are resident endothelial cells from the particular tissue being modeled. (e.g., lung microvascular endothelial cells to model a lung-like niche).

A method for screening comprising the steps of: (a) forming a microvascular niche model; (b) adding patient-derived tumor cells to the formed microvasculature niche model; (c) allowing the tumor cells to become dormant; and (d) screening for molecules of interest that have therapeutic efficacy against dormant tumor cells. In various embodiments, the molecules of interest are small molecules, peptides, antibodies, siRNAs, or antisense molecules.

A method for screening comprising the steps of: (a) forming a microvascular niche model; (b) adding patient-derived tumor cell lines to the formed microvasculature niche model; (c) allowing the tumor cells to become dormant; and (d) screening for small molecules, peptides, antibodies, siRNAs, other compounds or molecules, etc. that sensitize dormant tumor cells to chemotherapeutic agents, radiation, targeted agents (e.g., Herceptin), or any combination thereof.

A method for screening comprising the steps of: (a) forming a microvascular niche model seeded with cells of interest, wherein the seeded cells of interest are localized tumor cells from a patient that are seeded onto the formed microvasculature niche model; (b) determining at various time points if any growth of the tumor cells occurs to assess the capacity of a patient's tumor for dormancy or metastatic colonization. In other embodiments, the method further comprising step (c) contacting a drug or therapeutic with said cells in said microvasculature niche model to assess the efficacy of a particular drug or therapeutic compound against a patient's tumor cells.

A method for screening comprising: (a) forming a microvascular niche model seeded with cells of interest; (b) administering compounds to the seeded cells; (c) profiling the RNA or protein levels of the cells of interest grown in the microvascular niche; (d) comparing the RNA or protein profiles between microvascular niche versus stroma alone; and (e) identifying compounds that drive tumor cells into a dormant state.

A method for screening comprising the steps of: (a) forming microvascular niche models with different densities of neovascular tips seeded with cells, (b) administering molecules of interest to the seeded cells; (c) profiling the RNA or protein levels of the cells of interest grown in the microvascular niche; (d) comparing the RNA or protein profiles between microvascular niches with different tip densities; and (e) identifying molecules of interest with pro-metastatic functions.

In another embodiment, we have identified factors that mediate these two states—a dormancy-inducing niche (mediated by stable microvasculature via thrombospondin-1) as well as a tumor-promoting niche (mediated by sprouting neovasculature through active TGF-beta1 and periostin). Thus, this model can be used to identify potentially novel factors that mediate tumor quiescence and outgrowth using—OMICS technologies.

### BRIEF DESCRIPTION OF THE FIGURES

FIGS. 1A-1H: Dormant breast tumor cells reside on microvascular endothelium in distant tissues in vivo. FIG. 1A is a schematic showing GFP-Luc MDA-MB-231 cells how were injected into the inguinal mammary gland of NOD-SCID mice. Tumors were resected at 3 wks (V_{avg}=0.5 cm³; representative bioluminescence shown). Mice that were relapse-free after 6 wks (4/20 mice) were sacrificed and visceral organs were dissected. FIG. 1B is representative image of a primary tumor section fixed and stained for endothelial-specific marker CD31 (red), and cell cycle marker Ki67 (white). DNA was labeled with Hoechst 33342 (blue) (colors shown in grayscale). FIG. 1C is an image showing dormant (Ki67-negative) DTCs (white asterisks) found residing on microvascular endothelium in lungisolated from mice sacrificed 6 wks after primary tumor resection. FIG. 1D is an image showing dormant (Ki67-negative) DTCs (white asterisks) found residing on microvascular endothelium in BoMa tissues isolated from mice sacrificed 6 wks after primary tumor resection. FIG. 1E is a schematic illustrating mCherry T4-2 cells (false-colored green but shown in grayscale here for consistency) introduction via intra-cardiac injection. Mice that did not show any evidence of metastatic burden were sacrificed 8 wks later. FIG. 1F is an image showing dormant (Ki-67 negative) T4-2 BCCs (white asterisk) found residing perivascularly in lung. FIG. 1G is an image showing dormant (Ki-67 negative) T4-2 BCCs (white asterisk) found residing perivascularly in bone marrow. FIG. 1H is an image showing dormant (Ki-67 negative) T4-2 BCCs (white asterisk) found residing perivascularly in brain. Scale bars=20 μm.

FIGS. 2A-2J: Microvascular endothelium induces sustained quiescence of breast tumor cells in engineered cultures. FIG. 2A is a schematic showing lung and BoMa stroma (LFs and MSCs, respectively) seeded alone or with mCherry-E4-ECs. In co-culture, mCherry-E4-ECs self-assembled into 3D microvascular networks over 7 d. YFP-expressing BCCs (T4-2) were then seeded sparsely (240/cm²) in SFM onto stroma or microvascular niche cultures and overlaid with a drip of laminin-rich ECM (LrECM) diluted in media to provide BCCs with a 3D microenvironment (See Lee, G. Y., Kenny, P. A., Lee, E. H. & Bissell, M. J. Three-dimensional culture models of normal and malignant breast epithelial cells. Nat Methods 4, 359-365 (2007)). Entire wells were imaged 10 days later. FIG. 2B are representative images of T4-2 cell growth within lung-like or BoMa-like niches containing stroma only, or stroma+ECs, 10 days post-seeding. Scale bars=500 μm. FIG. 2C is a graph of tumor cell area fraction of YFP T4-2 at day 10 (normalized by value measured immediately post-seeding to correct for any minor variations in initial seeding density) in lung-like niches. FIG. 2D is a graph of tumor cell area fraction of YFP T4-2 at day 10 (normalized by value measured immediately post-seeding to correct for any minor variations in initial seeding density) in BoMa-like niches. n=5 sets of co-cultures analyzed per condition. Error bars denote s.e.m. **p=0.001 and ***p<0.0001 by two-tailed t test. Day 10 co-cultures were fixed and stained for CD31 to label ECs and Ki67 to identify actively cycling tumor cells (FIG. 2B, inset; Scale bar=50 μm). FIG. 2E is a graph of the percentage of Ki67-negative clusters (white asterisk in FIG. 2B, inset) quantified for T4-2 cells seeded on lung-like niches. FIG. 2F is a graph of the percentage of Ki67-negative clusters (white asterisk in FIG. 2B, inset) quantified for T4-2 cells seeded on BoMa-like niches n=5 sets of co-cultures analyzed per condition. Error bars denote s.e.m. ***p<0.0001 by two-tailed t test. FIG. 2G is a graph of tumor cell growth measured over an additional 7 days (day 17 normalized by day 10) in lung-like (niches to determine whether quiescent tumor clusters at day 10 remained quiescent. FIG. 2H is a graph of tumor cell growth measured over an additional 7 days (day 17 normalized by day 10) in BoMa-like niches to determine whether quiescent tumor clusters at day 10 remained quiescent. n=5 sets of co-cultures analyzed per condition. Error bars denote s.e.m. ***p<0.0001 by two-tailed t test. FIG. 2I is a collection of live images of representative T4-2 cells on lung-like stroma and microvascular niche for day 10 and day 17, with IF staining to confirm Ki67 status. FIG. 2J is a collection of live images of representative T4-2 cells on BoMa-like stroma and microvascular niche cultures. Note that stroma culture scale bars=100 μm and microvascular niche culture scale bars=50 μm.

FIGS. 3A-3J: Thrombospondin-1 is an angiocrine tumor suppressor. Lung- and BoMa-like stroma and microvascular niche cultures were decellularized and residual proteins were acid extracted and subjected to LC-MS/MS analysis. FIG. 3A is a heatmap of ECM proteins (spectral counts) from lung-like microvascular niche (LF+EC) normalized by lung stroma (LF; sorted high to low) and BoMa-like microvascular niche (MSC+EC) normalized by BoMa stroma (MSC). Logₑ intensity scale shown at lower left. FIG. 3B is an image showing localized expression of TSP-1 at the interface between dormant DTCs and lung microvasculature in spontaneous metastasis models (white arrowhead). FIG. 3C is an image showing localized expression of TSP-1 at the interface between dormant DTCs and lung microvasculature in experimental metastasis models (white arrowhead). Scale bars=10 μm. FIG. 3D is an image showing TSP-1 localization to the vascular BM in non-tumor bearing mice in lung. FIG. 3E is an image showing TSP-1 localization to the vascular BM in non-tumor bearing mice in bone. FIG. 3F is an image showing TSP-1 localization to the vascular BM in non-tumor bearing mice in brain. Scale bars: 10 μm for lung, 20 μm for others. FIG. 3G is an image showing endothelial source of TSP-1 by utilizing a 3D model of capillary morphogenesis where sprouting ECs are separated from inductive LFs by several millimeters. TSP-1 colocalized with type IV collagen in the BM of established microvessel stalks (white arrowheads), but TSP-1 appeared to be downregulated at neovascular tips (white asterisks). Scale bar=50 μm. This was confirmed in FIG. 3H, a graph showing the quantification of TSP-1 intensity at stalks vs. tips (n=16 microvessels pooled from 3 different experiments). Error bars denote s.e.m. ***p=0.0005 by two-tailed paired t-test. FIG. 3I is a graph showing add-back of TSP-1 to T4-2 cells plated on lung stroma effectively substituted for the presence of ECs by causing a significant reduction in tumor cell growth (normalized to vehicle condition; n=3 sets of co-cultures analyzed per condition). Error bars denote s.e.m. *p<0.05 when compared to Vehicle by one-way ANOVA and Dunnett's multiple comparisons test. FIG. 3J is a graph showing treated lung-like microvascular niches with a TSP-1 blocking antibody have significantly enhanced tumor cell growth (vs. IgG control; n=5 sets of co-cultures analyzed per condition). Error bars denote s.e.m. **p=0.0054 by two-tailed unpaired t-test.

FIGS. 4A-4D: Opposite regulation of tumor dormancy and growth by endothelial sub-niches: stable endothelium inhibits—whereas neovascular tips promote—breast tumor cell growth. FIG. 4A, inset i, is an immunofluorescence image of YFP T4-2 on BoMa-like microvascular niche after 10 d. *: Ki67-negative tumor cluster; T: neovascular tips surrounding proliferative tumor. Scale bar=100 μm. FIG. 4A insets ii-iv are additional examples of Ki67-negative BCCs residing on/near microvasculature from that same culture shown in inset i. Scale bar=50 μm. FIG. 4B is a collection of still images from live tracking of histone H2B-GFP T4-2 cells on BoMa-like microvascular sub-niches (stable endothelium and neovascular tip) from 0-72 h (see also Supplemental Movies 1-2). White arrowheads denote approaching tip and subsequent T4-2 division. Scale bars: left-most panel=100 μm, right panels=50 μm. FIG. 4C is a scatter plot of T4-2 cell dwell time fraction (t_{dwell}/t_{div}) within stable (t_{dwell, stable})/neovascular (t_{dwell, neo}) or stromal (t_{dwell, stroma}) sub-niches vs. division time (t_{div}, n=229 cells pooled from 3 separate time-lapse experiments). Pearson correlation analysis yielded significant correlation of t_{dwell, stable} with t_{div}, and significant anti-correlation of t_{dwell, neo} with t_{div}, meaning that BCCs with longer division times tended to dwell more around stable endothelium and less around neovascular tips. Pearson coefficients (r) for stable endothelium, neovascular tips, and stroma are listed to right of plot. *p=0.014 and **p=0.001 (two-tailed). FIG. 4D is a graph of dwell time (% of time to first division) as a function of sub-niche for fastest dividing tumor cells (t<t_{avg}−SD; n=30 cells) and slowest dividing tumor cells (t>t_{avg}+SD; n=59 cells).

FIGS. 5A-5F: Notch1-mediated reduction in neovascular tips suppresses breast tumor cell outgrowth. Microvascular niches were created with stromal cells mixed with shCtrl E4-EC and/or shNotch1 E4-EC. YFP T4-2 cells were then seeded in SFM and growth was analyzed 10 days later. FIG. 5A are images of microvascular niches composed of shCtrl E4-EC, shNotch1 E4-EC, or a 1:1 mix of the two (‘sh1:1’) fixed and stained for CD31 at day 7. Scale bar=200 μm. FIG. 5B is a graph of neovascular tip number/field (large white dots in FIG. 5a) and FIG. 5C is a graph of branch point density (small yellow dots in FIG. 5A) (n=15 fields of microvascular networks pooled from 5 separate co-cultures). Error bars denote s.e.m ***p<0.001 when compared to shCtrl condition by one-way ANOVA and Dunnett's post-test. FIG. 5D are representative images of YFP T4-2 seeded upon shCtrl EC, shNotch1 EC, or sh1:1 cultures, fixed and stained for CD31 and Ki67 10 d post-seeding. Scale bar=200 μm.

FIG. 5E is a quantification of normalized tumor cell area fraction and FIG. 5F is a quantification of % Ki67-negative clusters (each normalized by shCtrl condition; n=5 sets of co-cultures analyzed per condition). Error bars denote s.e.m. *p<0.05 when compared to shCtrl condition by one-way ANOVA and Dunnett's post-test in FIG. 5E.

FIGS. 6A-6G. Ectopic vascular sprouting promotes growth of injected breast tumor cells in zebrafish larvae. FIG. 6A is an experimental schematic: ˜1-10 mCherry-MDA-MB-231 BCCs were injected into the subintestinal space of 3.5 dpf mtp−/− mutant zebrafish and WT siblings (both containing the fli1:eGFP transgene) and imaged 4 days later. FIG. 6B is an image showing WT subintestinal vessels had few sprouts by the injection time point 3.5 dpf, while FIG. 6C is an image showing that the ectopic sprouting phenotype of the mtp−/− mutant was exaggerated (gray asterisks denote neovascular sprouts). Scale bar=50 μm. FIG. 6D is a quantification of ectopic/neovascular sprouts in subintestinal space of WT and mtp−/− mutant siblings (n=25 WT zebrafish analyzed; n=23 mtp−/− zebrafish analyzed). Error bars denote s.e.m. ***p<0.0001 by two-tailed unpaired t-test. FIG. 6E is a representative image of WT zebrafish 4 days post-injection (i.e., 7.5 dpf) with mCherry-MDA-MB-231 cells. FIG. 6F is a representative image of mtp−/− mutant zebrafish 4 days post-injection (i.e., 7.5 dpf) with mCherry-MDA-MB-231 cells. Scale bar=100 μm. White arrow in FIG. 6E points to small cluster on abluminal surface of subintestinal vessel of WT, while white arrows in FIG. 6F point to larger clusters localized to neovascular tips in mtp−/− mutant. FIG. 6G is a graph showing tumor cell area fraction in the subintestinal space at 7.5 dpf and normalized to the corresponding value post-injection for each surviving zebrafish with viable tumor cells in its subintestinal space to account for any variations in injection density (n=16 WT zebrafish analyzed; n=9 mtp−/− zebrafish analyzed). Error bars denote s.e.m. **p=0.005 by Mann-Whitney test.

FIGS. 7A-7I. Neovascular tips comprise ‘micrometastatic niches’ enriched for POSTN and TGF-β1. FIG. 7A is a heatmap of ECM proteins (spectral counts) from 1) neovascular tip^{high} cultures (LF+shCtrl EC) normalized by lung stroma (LF), 2) neovascular tip^{low} cultures (LF+shNotch1 EC) normalized by lung stroma (LF), and 3) tip^{high} cultures normalized by tip^{low} cultures (sorted high to low with respect to this comparison, log₂ scale). FIG. 7B is a representative image of microvessels stained for POSTN. FIG. 7C is a representative image of microvessels stained for active TGF-β1. FIG. 7D is a representative image of microvessels stained for latent TGF-β1. Scale bar=20 μm. FIG. 7E is a quantification of relative POSTN (left) and active TGF-β1 intensity (right) at the tip vs. stalk of microvessels (n=15 microvessels were pooled from 3 different experiments for analysis of POSTN intensity quantification; n=16 microvessels were pooled and analyzed for active TGF-β1 intensity quantification). Error bars represent s.e.m. ***p<0.0001 by paired two-tailed t-test. FIG. 7F is a representative image of microvascular niche cultures seeded with T4-2 cells and treated with vehicle twice over the first 48 h, and imaged at day 10. FIG. 7G is a representative image of microvascular niche cultures seeded with T4-2 cells and treated with a combination of POSTN (50 ng/ml) and TGF-β1 (10 pg/ml) twice over the first 48 h, and imaged at day 10. Scale bar=500 μm. FIG. 7H is a graph showing normalized tumor cell area fraction of YFP T4-2 at day 10 treated by vehicle or by said combination of POSTN and TGF-β1 (‘combo’; n=5 sets of co-cultures analyzed per condition). Error bars represent s.e.m. ***p<0.0001 by two-tailed t test. FIG. 7I is a visual summary of the findings: In distant microenvironments, single or small clusters of DTCs reside in the perivascular niche and are maintained in a quiescent state by endothelial-derived factors. Here, we have identified TSP-1 as one such factor, while perlecan was identified by others as an EC-derived factor that suppresses tumor growth (Franses, J. W., Baker, A. B., Chitalia, V. C. & Edelman, E. R. Stromal endothelial cells directly influence cancer progression. Sci Transl Med 3, 66ra65 (2011)). Other ECM molecules such as laminins, type IV collagen and latent TGF-β binding proteins (LTBPs) may contribute directly or indirectly to the dormant niche. As vascular homeostasis is disrupted with induction of neovascular sprouting, endothelial architecture is perturbed. The result is not only loss of suppressive signals (e.g., TSP-1), but deposition of ECM molecules and growth factors that promote micrometastatic outgrowth. Thus, maintaining vascular homeostasis could be the key to sustaining DTC dormancy long-term.

FIG. 8A-8C: E4ORF1 mediates survival and functional differentiation of endothelial cells in serum- and cytokine-free conditions. FIG. 8A is an image comparison of wild-type (wt)- and E4ORF1-HUVEC₂₄ survival on tissue culture plastic after 48 h of culture within SFM. Scale bar=100 μm. FIG. 8B is an image showing wt-(left) and E4-ECs (right) after 24 h culture atop of LrECM in serum- and cytokine-free conditions. Scale bar=100 μm. FIG. 8C is an image showing wt-(left) and E4-ECs (right) in co-cultures with LFs were allowed to form microvascular networks for 7 d in growth medium prior to washout and addition of serum- and cytokine-free medium for an additional 10 d. Scale bar=100 μm.

FIGS. 9A-9E: Microvasculature suppresses growth of luminal, ER₊ BCCs as well as high metastatic, triple-negative BCCs. FIG. 9A is representative images of YFP-MCF-7 growth within lung-like niches containing only stroma or stroma+ECs (‘microvascular niches’) after 10 d. Scale bar=500 μm. FIG. 9B is a quantification of YFP-MCF-7 area fraction in each of these conditions (n=5 sets of co-cultures analyzed per condition). Error bars represent s.e.m. ***p<0.0001 by two-tailed t test. FIG. 9C is representative images of YFP-MDA-MB-231 BCCs after 10 d on lung-like niches containing only stroma or stroma+ECs. Scale bar=500 μm. FIG. 9D is a quantification of YFPMDA-MB-231 area fraction in each of these conditions (n=5 sets of co-cultures analyzed per condition). Error bars represent s.e.m. p=0.09 by two-tailed t test. FIG. 9E is representative images of lung-like stroma and microvascular niche cultures seeded with YFP-MDA-MB-231, fixed 10 d later and stained for CD31 (red) and Ki67 (green). Note on right that Ki67-negative MDA-MB-231s are located on microvasculature. Scale bar=50 μm.

FIGS. 10A-10C: Medium conditioned by microvascular niche cultures does not substitute for presence of microvasculature. FIG. 10A is a representative image of YFP-T4-2 cells cultured on lung-like microvascular niche for 10 d, fixed and stained for CD31 (red) and Ki67 (green; yellow mark towards center of image is debris). Scale bar=200 μm. Inset shows corresponding field at time of seeding. Note that all tumor cell clusters (white arrow heads) appear to be derived from single tumor cells (white dotted circles, inset). The presence of a large, proliferative tumor cluster at the culture's edge (white arrow) hinted that the angiocrine tumor suppressor(s) was not a freely diffusible molecule. Conditioned media (CM) from established LF cultures or lung microvascular niches (LF+EC) were added daily to cultures consisting of LFs seeded with YFP-T4-2 cells. FIG. 10B is representative images of tumor growth after 7 days of CM treatment. Scale bar=500 μm. FIG. 10C is a quantification of normalized tumor area cell fraction at day 7 (n=5 sets of cocultures analyzed per condition). Error bars represent s.e.m. ‘NS’ denotes no significance by two-tailed unpaired t test.

FIG. 11: Validation of a shRNA clone that significantly reduced endothelial cell Notch1 expression at the protein level. FIG. 11 is an image of a representative immunoblots for Notch1 (intracellular domain (ICD) detected, top) and the nuclear membrane protein Lamin A/C (bottom). Values correspond to knockdown achieved with each shRNA clone after normalizing to Lamin A/C using band densitometry. Clone sh8393 was used for all experiments presented in FIG. 5.

FIGS. 12A-12G: Enriching naturally for neovascular tips promotes outgrowth of breast tumor cells. FIG. 12A is an image of lung-like microvascular niche culture fixed at day 3 of network development and stained for CD31 (light gray microvascular structures) and Hoechst 33342 (gray dotted structures) to label DNA. FIG. 12B is an image of lung-like microvascular niche culture fixed at day 7 of network development and stained for CD31 (light gray microvascular structures) and Hoechst 33342 (gray dotted structures) to label DNA. Neovascular tips are labeled with white dots. Scale bars=200 μm. FIG. 12C is an image of microvascular niche culture seeded with T4-2 BCCs at day 3 and assessed after 10 days. FIG. 12D is an image of microvascular niche culture seeded with T4-2 BCCs at day 7 and assessed after 10 days. Scale bar=200 μm. FIG. 12E is a quantification of neovascular tips at day 3 of network development vs. day 7 of network development (n=15 fields of microvascular networks pooled from 5 separate cocultures). Error bars denote s.e.m. ***p<0.0001 when compared by two-tailed t test. FIG. 12F is a graph showing T4-2 tumor cell area fraction 10 days after seeding microvascular networks at either day 3 or day 7 of development (normalized to day 7 values; n=5 sets of co-cultures analyzed per condition). Error bars represent s.e.m. ***p<0.0001 when compared by two-tailed t test. FIG. 12G is a graph showing percentage of Ki67-negative T4-2 clusters 10 days after seeding microvascular networks that developed for 3 or 7 days (n=5 sets of co-cultures analyzed per condition). Error bars represent s.e.m. *p=0.016 when compared by two-tailed t test.

FIGS. 13A-13F: In vivo validation of POSTN and TGF-β1 expression around endothelial tip cells in physiologic and pathologic contexts. FIG. 13A is a schematic of whole-mounted neonatal (postnatal day 5, P5) retina and FIG. 13B is a schematic of brain metastasis sections used to analyze expression of identified tip cell-derived tumor promoters in physiologic and pathologic contexts. FIG. 13C is an image showing POSTN deposited by endothelial tip cells in the developing retina (white arrow). FIG. 13D is an image showing POSTN deposited by endothelial tip cells within brain metastases (white arrow). POSTN is expressed sporadically on established phalanx endothelium within the retina (FIG. 13C, inset), and is absent from endothelium on the contralateral side of the brain (‘normal’ tissue; FIG. 13D, inset). FIG. 13E is an image showing active TGF-β1 expressed in the immediate vicinity of endothelial tip cells in the developing retina (white arrow). FIG. 13F is an image showing active TGF-β1 expressed in the immediate vicinity of endothelial tip cells within brain metastases (white arrow). Conversely, active TGF β1 is absent around retinal phalanx endothelium (FIG. 13E, inset) and is expressed randomly around microvasculature on the contralateral side of the brain (FIG. 13F, inset). Scale bars=20 μm.

### DETAILED DESCRIPTION OF THE INVENTION

In some embodiments, the present model provides for in vitro organotypic modeling of microvascular niches from various tissues. In various embodiments, the present models comprised of a specific stromal cell type of the endothelial cells and either endothelial cells from the particular tissue or human umbilical endothelial cells (HUVEC).

As shown in the Examples, we first localized dormant DTCs from the breast to the microvasculature of the lung, bone marrow and brain, and then constructed organotypic models of lung- and bone marrow-microvascular niches—that are maintained in serum- and cytokine-free conditions—in order to demonstrate that endothelium directly regulates tumor dormancy. We have discovered that stable endothelium induces tumor quiescence whereas disruption of stable endothelium accelerates tumor outgrowth.

In various embodiments, the models may be used to provide the microvascular niches modeling the most common tissue sites of relapse in cancer. In other embodiments, the models are used to model any tissue site in the body

In various embodiments, stromal cells and endothelial cells are first cultured together to allow self-assembly and formation of three-dimensional (3D) microvascular niches. In various embodiments, the stromal cells are resident stromal and/or mesenchymal cells which when used, model the tissue and vasculature in specific organs. Table 1 below provides a non-limiting list of resident stromal/mesenchymal cells which when cultured with endothelial cells then self-assemble and form the 3D microvascular niches described.

TABLE 1
Resident Stromal/Mesenchymal Cells Used to Model Specific Organs

| Organ | Resident Stromal/Mesenchymal Cell |
|---|---|
| Lung | Lung Fibroblast |
| Bone Marrow | Mesenchymal Stem Cell |
| Liver | Hepatic Stellate Cell |
| Brain | Brain Adventitial Fibroblasts |
| Skin | Skin Fibroblasts |
| Muscle | Skeletal Muscle Fibroblasts, Skeletal Muscle |
| | Stellate Cells, Skeletal Muscle Myoblasts |
| Heart | Cardiac Fibroblasts, Cardiac Myocytes |
| Pancreas | Pancreatic Stellate Cells |
| Prostate | Prostate Fibroblasts |
| Ovary | Ovary Fibroblasts |
| Lymph Node | Lymphatic Fibroblasts |
| Omentum | Omentum Fibroblasts |
| Breast | Mammary Fibroblasts |
| Spleen | Spleen Fibroblasts |
| Kidney | Renal Fibroblasts |
| Bladder | Bladder Stromal Fibroblasts |

Thus, a few illustrative examples from Table 1 include but are not limited to the following: In one embodiment, to form a lung tissue microvascular niche, lung fibroblasts, and HUVEC or lung endothelial cells can be used. In another embodiment, to form a bone marrow microvascular niche, mesenchymal stem cells, and HUVEC or bone marrow endothelial cells may be used. In another embodiment, to form a brain microvascular niche, human adventitial fibroblasts and astrocytes, and HUVEC or endothelial cells may be used. In yet another embodiment, to form a liver microvascular niche, liver stellate cells, and endothelial cells or HUVEC can be used.

In various embodiments, the endothelial cells can be isolated or selected from tissue using methods known in the art or described in the references below. In other embodiments, endothelial cells can be ordered from a commercial provider such as ScienCell or Lonza.

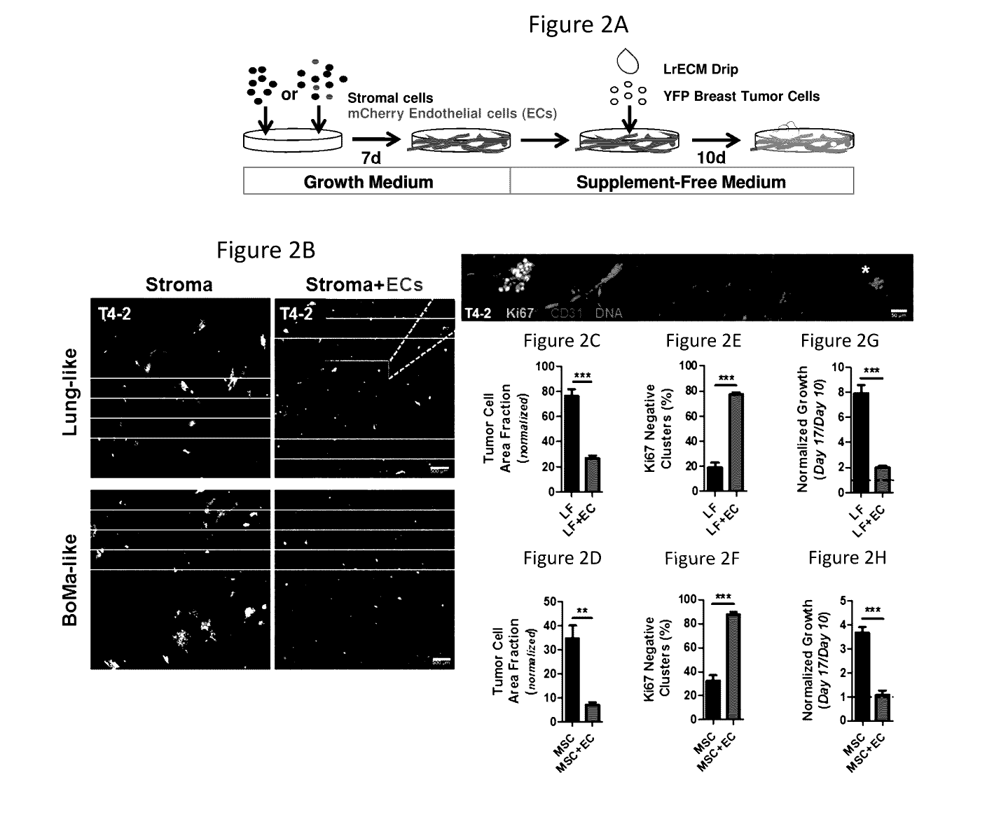

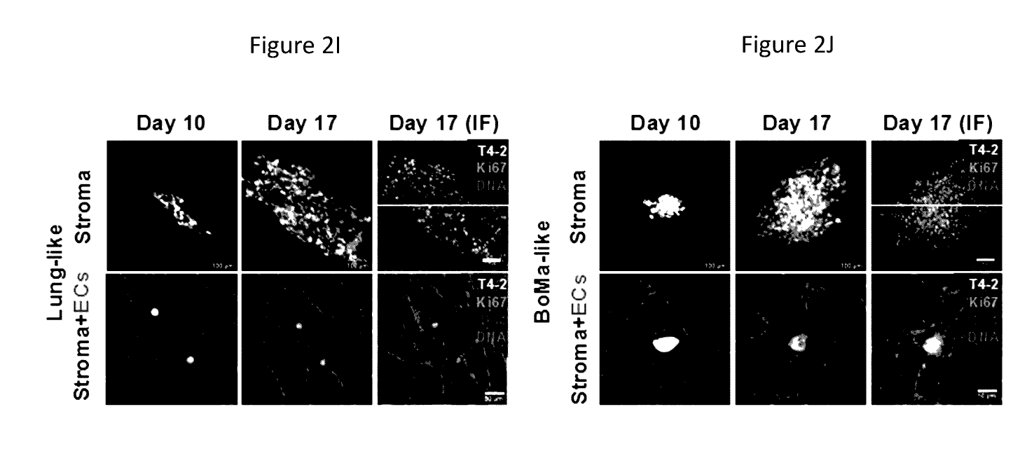

As shown in the schematic in FIG. 2A, the stromal cells and endothelial cells are then combined, and allowed to self-assemble and form three-dimensional microenvironments or complexes as shown in FIG. 2B. In various embodiments, methods for culturing and formation of the microvascular niches that can be used are as described in Evenson, L, et al., “Mural cell associated VEGF is required for organotypic vessel formation,” PLoS One. 2009 Jun. 4; 4(6):e5798, and in U.S. Pat. Nos. 7,244,576; 7,419,779; 7,485,414; 7,527,936; 7,566,546; and 8,574,827, all of which are hereby incorporated by reference in their entirety for all purposes.

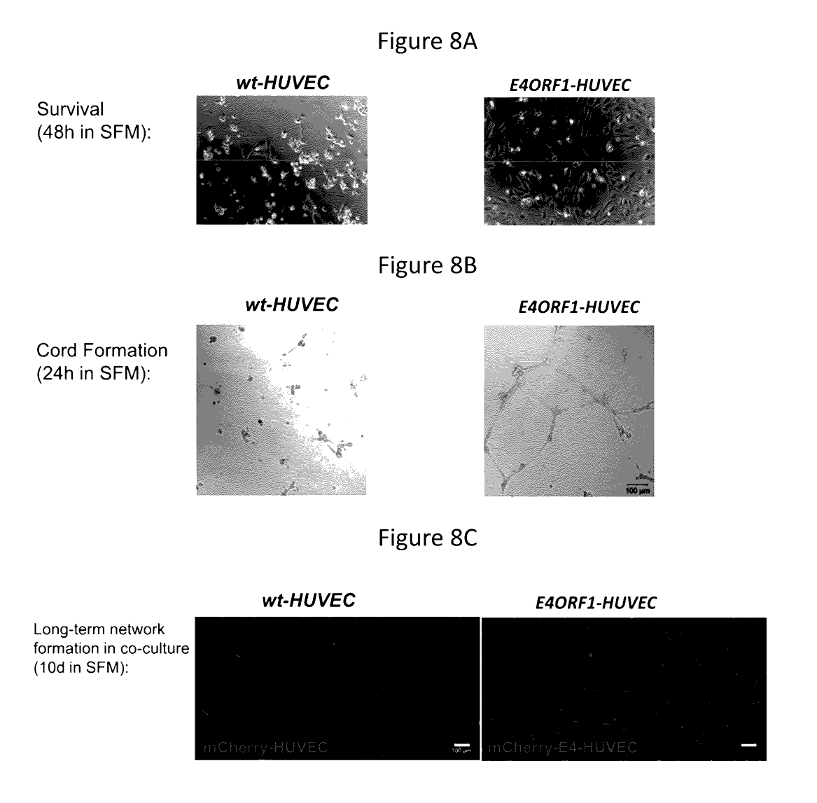

In other embodiments, the endothelial cells that the stromal cells are cultured with are human umbilical vein endothelial cells (HUVEC). In some embodiments, when HUVEC are used, the HUVEC are transduced with a lentiviral construct containing the human adenoviral E4ORF1 gene, which enables HUVECs to survive and form sustainable microvascular networks in Supplement-Free Medium (See FIG. 2A and FIGS. 8A, 8B, and 8C). Other methods and compositions useful for culturing endothelial cells stimulating angiogenesis are described in Zhang et al. (2004), J. Biol. Chem. 279(12):11760-66, U.S. Patent Pub. Nos. 20020051762, entitled “Purified Populations of endothelial stem cells,” and 20140045260, “Methods and Compositions for Promoting Survival and Proliferation of Endothelial Cells and Stimulating Angiogenesis,” and Int. Pub. No. WO/2008/089448, each of which is hereby incorporated by reference in their entirety for all purposes.

In some embodiments, the endothelial cells or HUVEC are transduced with an expression construct comprising a vector, reporter gene, and a gene, cDNA or nucleotide sequences that expresses an angiogenic or anti-angiogenic factors such as E4ORF1, VEGF, Thrombospondin-1, Notch1, Laminin, Nidogen-1 or -2, latent TGFB binding proteins, and collagen-4, etc, or antisense inhibitors of such angiogenic factors. Examples of cDNAs and angiogenic factors are described for example in Evenson, L, et al., “Mural cell associated VEGF is required for organotypic vessel formation,” PLoS One. 2009 Jun. 4; 4(6):e5798, and in U.S. Pat. Nos. 7,244,576; 7,419,779; 7,485,414; 7,527,936; 7,566,546; and 8,574,827, previously incorporated by reference in their entirety.

The expression vector usable in the present methods with the expression construct include pUC vectors (for example pUC118, pUC119), pBR vectors (for example pBR322), pBI vectors (for example pBI112, pBI221), pGA vectors (pGA492, pGAH), pNC (manufactured by Nissan Chemical Industries, Ltd.). In addition, virus vectors can also used including but not limited to lentiviral, adenoviral, retroviral or sendai viral vectors. The terminator gene to be ligated may include a 35S terminator gene and Nos terminator gene.

The expression system usable in the methods described herein include any system utilizing RNA or DNA sequences. It can be used to transform transiently or stably in the selected host (bacteria, fungus, plant and animal cells). It includes any plasmid vectors, such as pUC, pBR, pBI, pGA, pNC derived vectors (for example pUC118, pBR322, pBI221 and pGAH). It also includes any viral DNA or RNA fragments derived from virus such as phage and retro-virus derived (TRBO, pEYK, LSNLsrc). Genes presented in the invention can be expressed by direct translation in case of RNA viral expression system, transcribed after in vivo recombination, downstream of promoter recognized by the host expression system (such as pLac, pVGB, pBAD, pPMA1, pGa14, pHXT7, pMet26, pCaMV-35S, pCMV, pSV40, pEM-7, pNos, pUBQ10, pDET3, or pRBCS.) or downstream of a promoter present in the expression system (vector or linear DNA). Promoters can be from synthetic, viral, prokaryote and eukaryote origin.

The expression cassette may include 5′ and 3′ regulatory sequences operably linked, for examples, to the reporter gene or the angiogenic factor gene. “Operably linked” is intended to mean a functional linkage between two or more elements. For example, an operable linkage between a gene and a regulatory sequence (i.e., a promoter) is functionally linked that allows for expression of the gene. Operably linked elements may be contiguous or non-contiguous. When used to refer to the joining of two protein coding regions, by operably linked is intended that the coding regions are in the same reading frame. The cassette may additionally contain at least one additional gene to be co-transfected into the organism. Alternatively, the additional gene(s) can be provided on multiple expression cassettes. Such an expression cassette is provided with a plurality of restriction sites and/or recombination sites for insertion of the gene sequence. The expression cassette may additionally contain selectable marker genes or a reporter gene to be under the transcriptional regulation of the regulatory regions.

The expression cassette will include in the 5′-3′ direction of transcription, a transcriptional initiation region (i.e., a promoter), translational initiation region, a polynucleotide of the invention, a translational termination region and, optionally, a transcriptional termination region functional in the host organism. The regulatory regions (i.e., promoters, transcriptional regulatory regions, and translational termination regions) and/or the polynucleotide of the invention may be native/analogous to the host cell or to each other. Alternatively, the regulatory regions and/or the gene may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

Where appropriate, the polynucleotides may be optimized for increased expression in the transformed organism. For example, the polynucleotides can be synthesized using preferred codons for improved expression.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassette can also comprise a selectable marker gene for the selection of transformed or modulated cells. Selectable marker genes are utilized for the selection of transformed or differentiated cells or tissues. Marker genes include genes encoding antibiotic resistance, such as those encoding neomycin phosphotransferase II (NEO) and hygromycin phosphotransferase (HPT). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54), and m-Cherry (Shaner et al., Nature Biotechnology 22: 1567-72). The above list of selectable marker genes is not meant to be limiting. Any selectable marker gene can be used in the present embodiments.

In one embodiment, an expression cassette comprising the nucleotide sequence operably linked to a promoter that drives expression of a selective agent, signal peptide or label in the host organism, and the expression cassette further comprising an operably linked polynucleotide encoding a selective agent, signal peptide or reporter.

In other embodiments, the construct used herein includes an inducible reporter gene, such as mCherry, GFP, YFP, etc. In one specific embodiment, HUVECs are transduced with a lentiviral construct containing the human adenoviral E4ORF1 gene and reporter gene, mCherry. This provides for E4ORF1-HUVECs (E4-ECs)-expressing mCherry self-assembling into robust three-dimensional (3D) microvascular networks over 7 days when cultured with fibroblasts from lung (LFs) or with BoMa mesenchymal stem cells (MSCs).

Self-assembly and formation of 3D microvascular niches occurs while the stromal cells and endothelial cells are cultured together in growth medium. In some embodiments, formation of the 3D microvascular niches are allowed to form for about or at least 3-10 days, and more preferably about 5-7 days. In various embodiments, the 3D microvascular niche and microenvironment models various tissues, including but not limited to, lung, brain, bone marrow, liver, lymph node, ovary, omentum, pancreas, skeletal muscle, heart, skin, bladder, breast, prostate, kidney, or bladder (see Table 1 above).

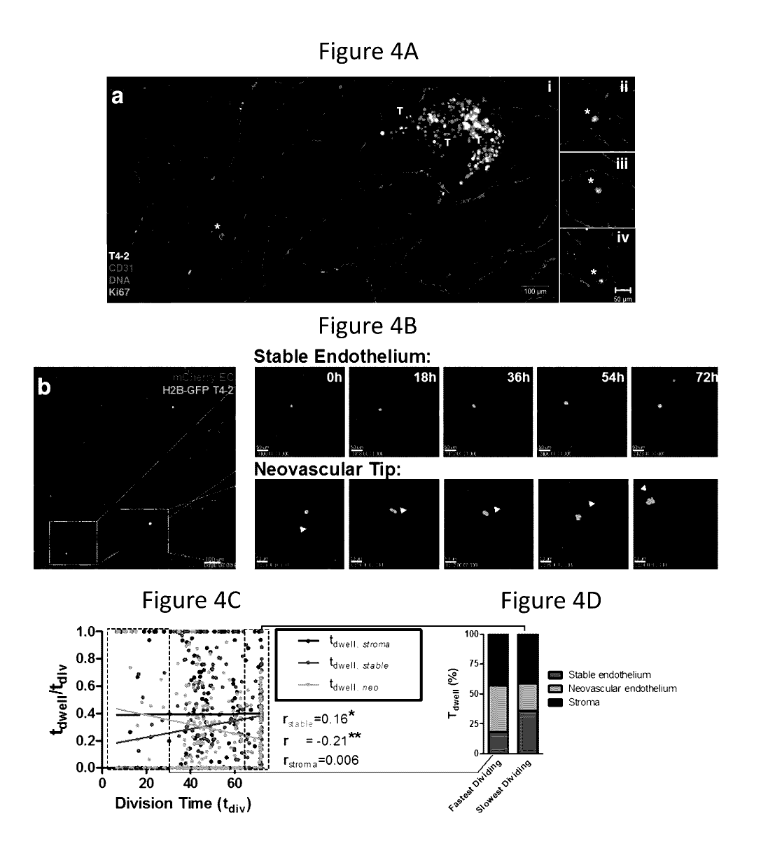

The formed 3D microvascular niches are then seeded or co-cultured with other cells. In some embodiments, the seeded cells and 3D microvascular niches are provided with medium and factors to provide and sustain a 3D microenvironment. For example, in one embodiment, the 3D microvascular niche is provided supplemental-free medium with a drip of laminin-rich ECM (LrECM) diluted in media to provide seeded breast cancer cells with a 3D microenvironment. The seeded cells are cultured in the 3D microenvironment for a sustained period. In some embodiments, the seeded cells are cultured for 7-15 days in the 3D microenvironment, more preferably 7-10 days. The seeded cells are observed or detected to determine their growth pattern. In various embodiments, observation of a stable growth pattern of seeded cells is an indicator that the seeded cells have adopted a quiescent or dormant state. See FIG. 4a. In other embodiments, observation of outgrowth or clustered cell growth indicates that the seeded cells are in a growth or possibly tumorigenic state. For example, if the seeded cells were biopsy sample breast cells from a patient, the observed growth state of the seeded biopsy breast cells indicates that the breast cells are indeed tumorigenic and possibly metastatic. Furthermore, if this outgrowth occurs in a particular 3D lung microvascular niche modeling a tissue such as lung, this could indicate that the cells if disseminated to lung tissue would enter a growth instead of dormant state.

3D microvascular niches can be seeded or cultured with other cells including but not limited cancer cells, cancer stem or progenitor cells, stem cells, progenitor cells, primary cells, other resident other resident cell types from the particular tissue being modeled (e.g., astrocytes or microglia for brain, epithelial cells, etc.), and/or non-resident cells (e.g., immune cells such as macrophages, B cells, T cells, other lymphocytes).

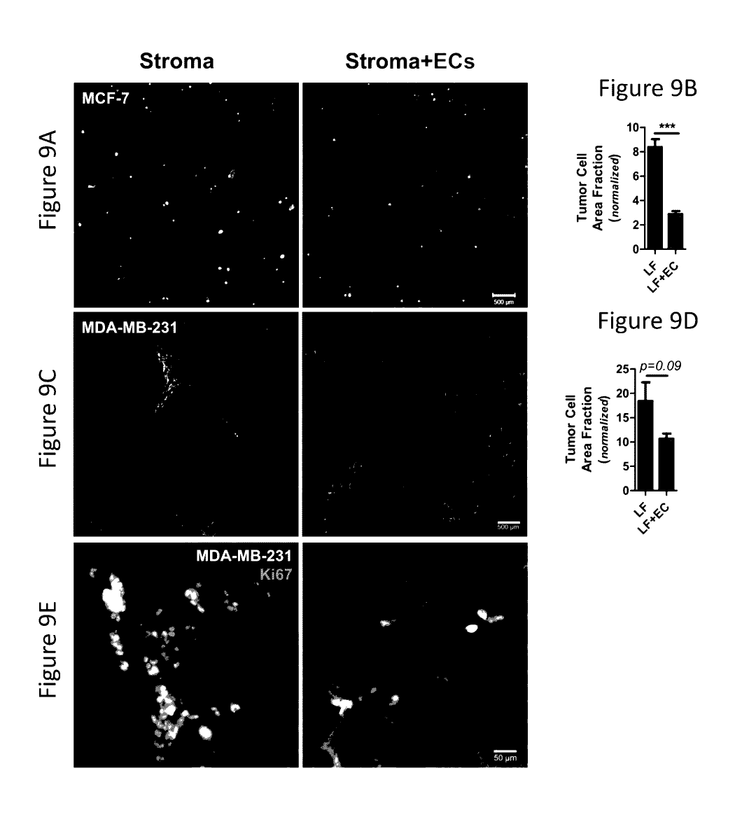

In other embodiments, the sample cells to be seeded with the 3D microvascular niches are cultured with a panel of microvascular niches which model the tissue where the sample are derived or obtained, and compared to the growth of sample cells seeded onto other 3D microvascular niches such as, lunch, bone marrow, brain, liver, lymph, etc. For example, the Examples describe yellow fluorescent protein (YFP)-expressing T4-2 cells seeded sparsely in SFM onto lung- and BoMa-like microvascular niches or onto only the corresponding stroma (i.e., LFs or MSCs) after an additional 10 days (FIG. 2a). T4-2 cells grew extensively on lung and BoMa stroma, growth of T4-2 cells on organotypic microvascular niches was reduced drastically (3-fold in lung-like- and 5-fold in BoMa-like-microenvironments; FIGS. 2B, 2C and 2D). Similar results were obtained also with a luminal, estrogen receptor-positive (ER⁺) BCC line (MCF-7) (FIGS. 9A and 9B).

Thus, the present methods provides for screening of cells. For example, cells obtained in a patient biopsy may be tested on three different organotypic microvascular niches as described herein and the observed growth or quiescence is detected and observed. Such observation can be used to inform a clinician as to the tumorigenicity or metastatic potential of the biopsied cells.

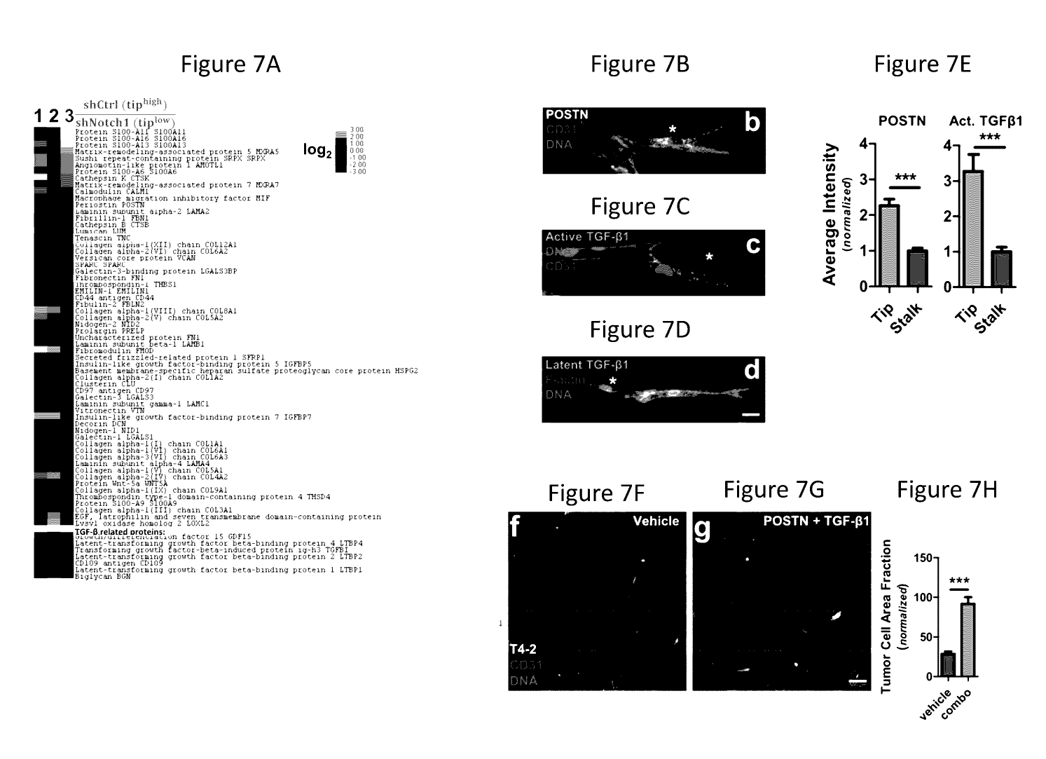

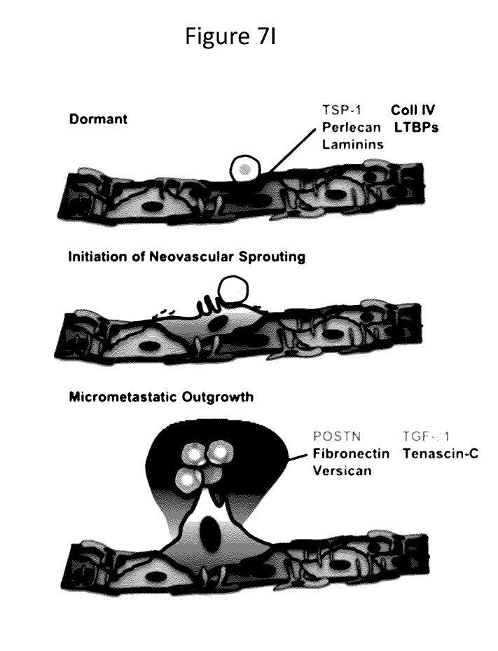

Microvasculature when stable prevents growth by excreting certain factors that restrain growth. We found that thrombospondin-1 is one of those restraining factors. When microvasculature is sprouting, the tips express tumor-promoting factors. Other likely repressive factors we found may be Laminin, Nidogen-1 or -2, latent TGFB binding proteins, and collagen-4. See the heat map in FIG. 7A.

Relevant sequence data for the protein, nucleic acids encoding thrombospondin, and related sequences include the nucleic acid GenBank accession number NM_0003246.2, SEQ ID NO:4; and protein GenBank accession number NP_003237, SEQ ID NO:5, hereby incorporated by reference.

Therefore, in various embodiments, methods of modulating angiogenesis in a subject, a method comprising the step of administering to the subject a therapeutically effective amount of a compound identified as a modulator of angiogenesis. In one embodiment, the subject is a human. In a further embodiment, the compound is an antibody, an antisense molecule, a small organic molecule, a peptide, or an RNAi molecule. In another embodiment, the compound inhibits angiogenesis. In another embodiment, a dormancy-inducing niche factor composition comprising a therapeutic amount of inducing agents of thrombospondin-1, Laminin, Nidogen-1 or -2, latent TGFB binding proteins, collagen-4, and/or combinations thereof.

In one embodiment, herein we describe methods to induce fully malignant, genotypically aberrant tumor cells into a state of sustained dormancy. We have scaled this model so that it is conducted in 96- and 384-well formats to be amenable to high-throughput and high-content screening. We envision that with cell culture robots, high-throughput screens testing arrays of compounds could be conducted in parallel and imaged in automated fashion in order to determine positive hits that either sustain tumor dormancy or disrupt tumor dormancy.

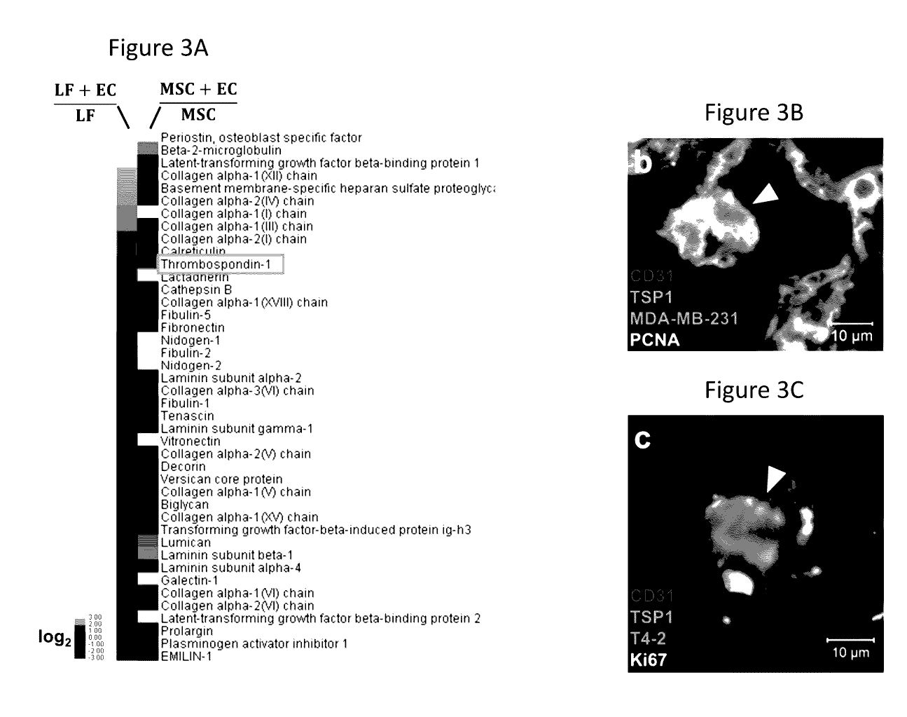

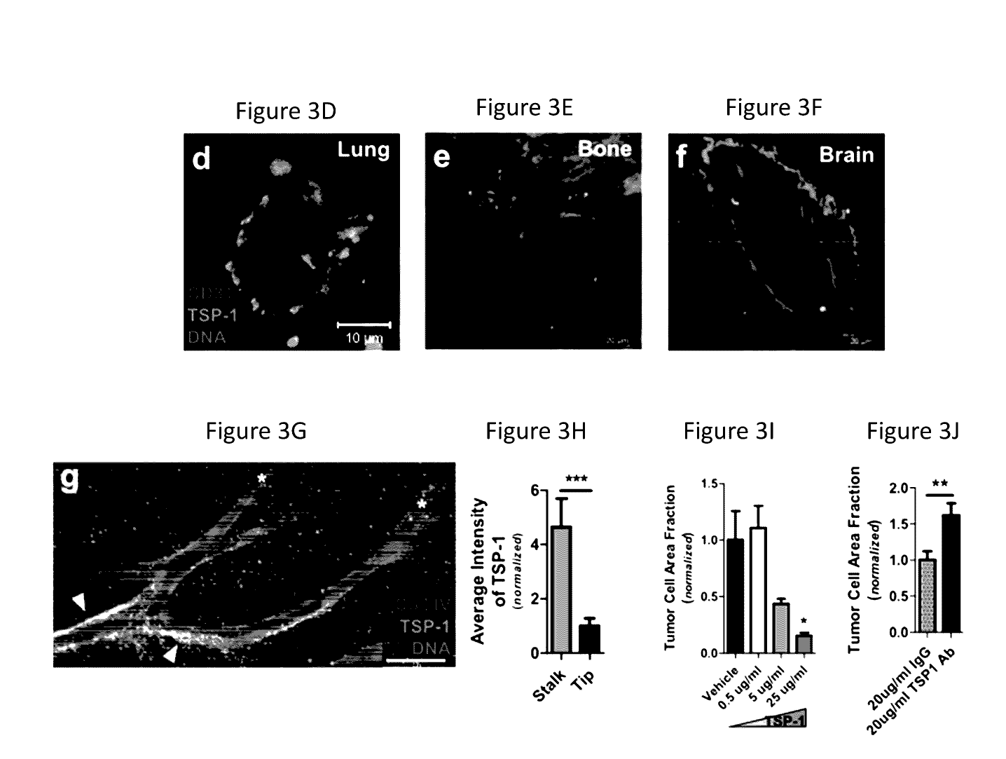

The present model may be used for screen, (e.g., in high-throughput), for drugs (e.g., using molecular compound libraries) that kill dormant cells, make dormant cells sensitive or susceptible to traditional chemotherapeutics like doxorubicin and paclitaxel (combinatorial therapeutic regimens), and/or agents that maintain dormancy long-term. An appealing and intriguing aspect of the model is that it contains functionally differentiated normal cell types, so there is an internal control for drug toxicity contained within the model itself. This model could also be used to screen drugs developed to target primary tumor or established metastases (e.g., anti-angiogenic therapies) to ensure that they do not disrupt the dormant niche and cause outgrowth of dormant cells. Lastly, of the factors identified via mass spectrometry (see FIG. 3a and FIG. 7a), some are poorly characterized and may result in identification of novel factors and/or pathways that can be targeted to induce tumor dormancy of slow-growing tumor cell populations.

In other embodiments, the model is used for its prognostic application. For instance, cells isolated from a patient's breast tumor could be cultured on organotypic niches of lung-, bone marrow- and brain-microvasculature. If the patient's cells were steered into a dormant state by 2 of these niches, but were resistant to the third (e.g., lung), this may be predictive of accelerated relapse specifically within the third resistant tissue (e.g., lung), and may inform and guide various or different treatment regimens. These are outstanding questions that could be answered specifically with our model, and potentially impact significantly how and when we treat metastatic disease.

In another embodiment, the present model provides for means to approximate growth kinetics by observing and tracking over tumor cell growth over time. One of several growth models (e.g., Gompertzian, etc) can be applied and then used to approximate what the growth kinetics (e.g., in vivo or in a patient) would be. For example, if cells obtained in a biopsy were applied to the present niche model in various tissues as described above, the cells can be allowed to grow for various time periods and growth observed. Day 10 to day 17 can be observed; any increase in growth would be cause for concern but no net growth would likely indicate that patient's cell are responding to the dormant niche of that organ.

In other embodiments, the present model may be used for in regenerative medicine and/or stem cell maintenance because stem cells are prone to reside perivascularly in a number of different organs. The present methods described herein and in the Examples may be used in conjunction with the present model to uncover novel molecules that maintain stem cell pluripotency, or the organotypic microvascular niches could simply be used to expand stem cell or other cell populations.

Not only are these engineered models powerful as a high-throughput screening tool, but they are also powerful tools that can be used in conjunction with—OMICS technologies (e.g., proteomics) in order to identify factors that characterize the dormant niche, induce tumor cells into a state of dormancy or draw them out of this state. Indeed, we have uncovered that our model has at least 2 different microenvironments, and already have identified factors that mediate these two states—a dormancy-inducing niche (mediated by stable microvasculature via thrombospondin-1) as well as a tumor-promoting niche (mediated by sprouting neovasculature through active TGF-beta1 and periostin). Thus, this model can be used to identify potentially novel factors that mediate tumor quiescence and outgrowth using—OMICS technologies.

In some embodiments, methods are provided for isolating or modulating cell populations having variable vascular tip growth. e.g., high or low tip growth. In various embodiments, such cell populations would allow for screening and selecting for novel factors which induce or inhibit cell dormancy, tip growth, angiogenesis, differentiation, growth and metastasis.

In another embodiment, methods for screening for molecules that induce dormancy, comprising the steps of: culturing stroma cells with endothelial cells and forming 3D microvasculature models of various tissues, seeding the culture with growing cells; applying a molecule of interest to induce dormancy in the seeded cells with the molecule of interest; detecting if dormancy is induced; culturing in a separate vessel stroma cells seeded with the growing cells; applying the molecule of interest; compare cell growth or dormant state in stroma without vasculature to stroma with vasculature formed.

In another embodiment, co-culture plates or kits providing the components required for engineering a dormancy model in a multi-well format for high-throughput culture, screening and assays.

### Examples

In a significant fraction of breast cancer patients, distant metastases emerge after years or even decades of latency. How disseminated tumor cells (DTCs) are kept dormant, and what ‘wakes them up’, are fundamental problems in tumor biology. To address these questions, we utilized metastasis assays in mice to show that dormant DTCs reside upon microvasculature of lung, bone marrow and brain. We then engineered organotypic microvascular niches to determine whether endothelial cells directly influence breast cancer cell (BCC) growth. These models demonstrated that endothelial-derived thrombospondin-1 induces sustained BCC quiescence. This suppressive cue was lost in sprouting neovasculature; time-lapse analysis showed that sprouting vessels not only permit, but accelerate BCC outgrowth. We confirmed this surprising result in dormancy models and in zebrafish, and identified active TGF-β1 and periostin as tumor-promoting, endothelial tip cell-derived factors. The present Examples reveal that stable microvasculature constitutes a ‘dormant niche,’ whereas sprouting neovasculature sparks micrometastatic outgrowth.

To determine whether endothelial cells and other factors deposited within their surrounding basement membrane were involved in the dormant niche, we utilized two mouse models of human breast cancer metastasis and discovered that dormant DTCs reside upon the microvasculature of lung, BoMa and brain. By creating organotypic models of lung- and BoMa-microvascular niches, we demonstrated that ECs induce and sustain BCC quiescence. Proteomic and functional analyses of proteins deposited in organotypic microvascular niches identified thrombospondin-1 TSP-1) as an endothelium-derived tumor suppressor. Importantly, TSP-1 was diminished near sprouting neovasculature, suggesting that tumors may escape growth regulation in this ‘sub-niche’. Time-lapse analysis confirmed that tumor growth was not just permitted, but in fact accelerated around neovascular tips, which we show are rich in tumor-promoting factors such as active TGF-β1 and periostin (POSTN). These findings establish a paradigm of differential regulation of DTC dormancy and relapse by distinct endothelial sub-niches, and suggest that preserving vascular homeostasis is critical to maintaining dormancy of DTCs.

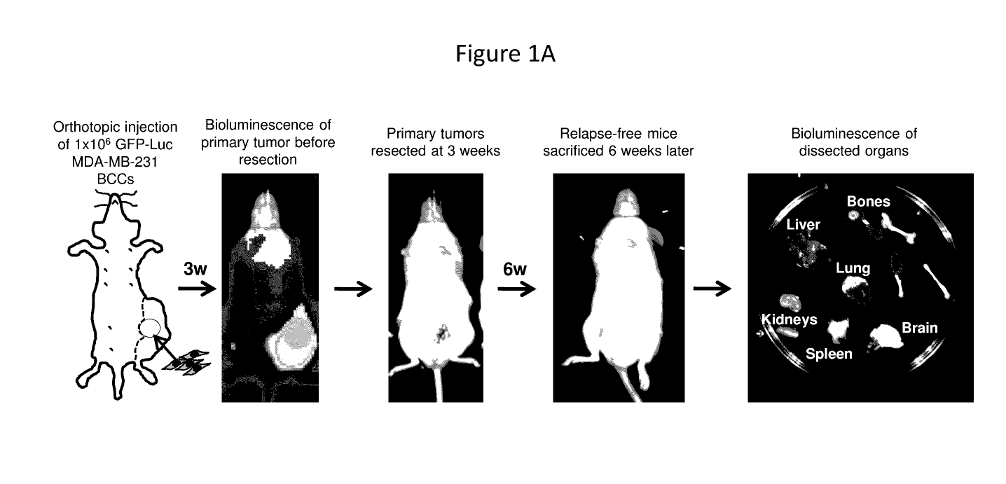

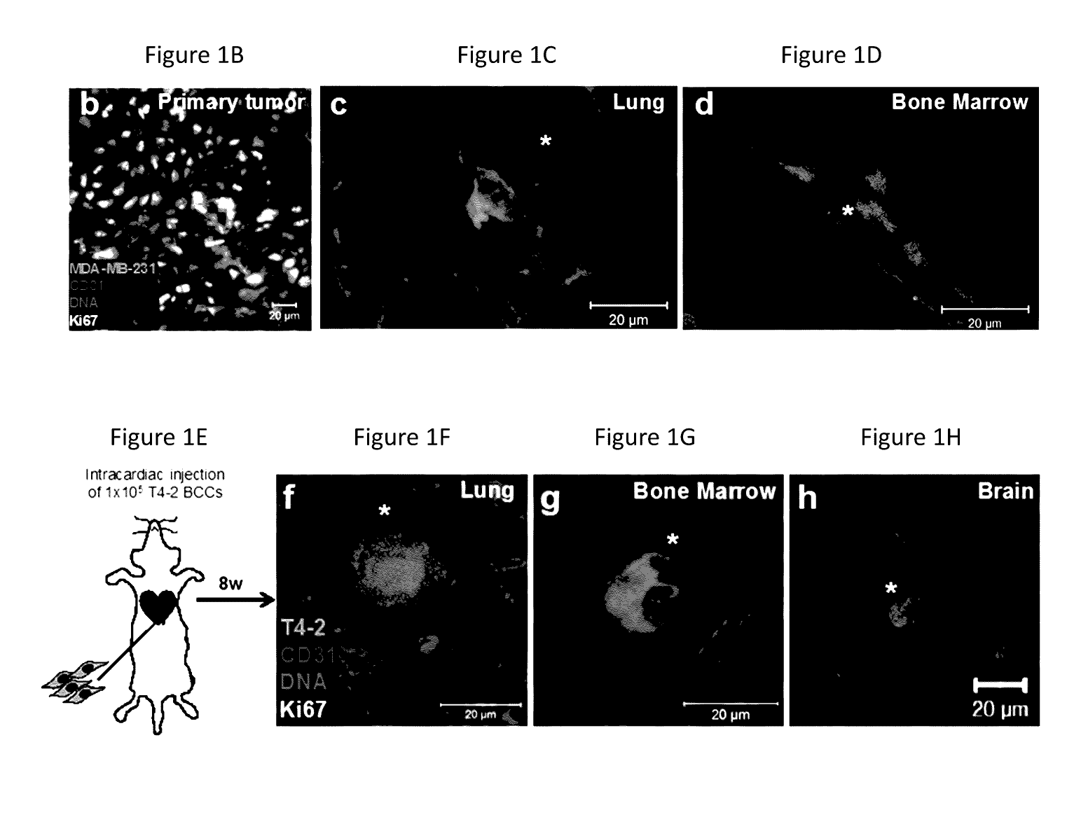

To determine whether dormant DTCs occupy a specific niche, we searched first for DTCs lacking expression of the cell cycle marker, Ki67 in a spontaneous metastasis model of breast cancer (See Francia, G., Cruz-Munoz, W., Man, S., Xu, P. & Kerbel, R. S. Mouse models of advanced spontaneous metastasis for experimental therapeutics. Nat Rev Cancer 11, 135-141 (2011)). Tumors resulting from orthotopic injection of MDA-MB-231, a bona fide metastatic BCC line expressing GFP-luciferase, were resected after 3 weeks (V_{avg}=0.5 cm³, FIG. 1A). Surviving mice that did not experience relapse at the primary site were sacrificed 6 weeks later. Bioluminescence of dissected visceral organs confirmed that BCCs disseminated to the canonical target organs lung, bone, liver, and brain (Paget, S. The distribution of secondary growths in cancer of the breast. 1889. Cancer Metastasis Rev 8, 98-101 (1989)) (FIG. 1a). In contrast to the resected primary tumors, in which BCCs proliferated actively whether nearby tumor vasculature or not (FIG. 1b), we found small clusters of GFP-positive/Ki67-negative BCCs residing directly on microvascular endothelium of both lung (FIG. 1c) and BoMa (FIG. 1d).

This observation was confirmed also with a weakly metastatic BCC line (mCherry-HMT-3522-T4-2, Briand, P., Nielsen, K. V., Madsen, M. W. & Petersen, O. W. Trisomy 7p and malignant transformation of human breast epithelial cells following epidermal growth factor withdrawal. Cancer Res 56, 2039-2044 (1996)), which was injected intra-cardially to facilitate dissemination to all target organs (FIG. 1e). Eight weeks after injection into the left ventricle of NOD-SCID mice, small clusters of mCherry-positive (false-colored green)/ki-67-negative T4-2 cells were found residing perivascularly in murine lung (FIG. 1f), BoMa (FIG. 1g) and brain (FIG. 1h). The consistent discovery of quiescent DTCs residing perivascularly 6 weeks after resection of the primary tumor in the first model, and 8 weeks post-injection in the second mode (suggested that endothelium might play an active role in regulating tumor dormancy.

Organotypic Microvascular Niches Demonstrate that Endothelial Cells Induce Sustained Quiescence of BCCs

Determining whether microvascular endothelium could directly influence tumor cell quiescence necessitated lung- and BoMa-Like designer microenvironments that would allow quantitative assessment of human BCC growth in the presence or absence of a microvascular network. There are considerable hurdles to engineering such models. For example, whereas ECs do not survive in serum- and cytokine-free medium (SFM), the addition of exogenous factors could mask the effects of EC-derived “angiocrine” factors on tumor growth (See Butler, J. M., et al. Endothelial cells are essential for the self-renewal and repopulation of Notch-dependent hematopoietic stem cells. Cell Stem Cell 6, 251-264 (2010); Seandel, M., et al. Generation of a functional and durable vascular niche by the adenoviral E4ORF1 gene. Proc Natl Acad Sci USA 105, 19288-19293 (2008); and Butler, J. M., Kobayashi, H. & Rafii, S. Instructive role of the vascular niche in promoting tumour growth and tissue repair by angiocrine factors. Nat Rev Cancer 10, 138-146 (2010)).

To overcome this limitation, primary human umbilical vein endothelial cells (HUVECs) were transduced with a lentiviral construct containing the human adenoviral E4ORF1 gene (Seandel, M., et al. Generation of a functional and durable vascular niche by the adenoviral E4ORF1 gene. Proc Natl Acad Sci USA 105, 19288-19293 (2008)), which enables HUVECs to survive and form sustainable microvascular networks in SFM (FIG. 8). E4ORF1-HUVECs (E4-ECs)-expressing mCherry self-assembled into robust three-dimensional (3D) microvascular networks (Evensen, L., et al. Mural cell associated VEGF is required for organotypic vessel formation. PLoS One 4, e5798 (2009)) over 7 days when cultured with fibroblasts from lung (LFs) or with BoMa mesenchymal stem cells (MSCs). We then compared the growth of yellow fluorescent protein (YFP)-expressing T4-2 cells seeded sparsely in SFM onto lung- and BoMa-like microvascular niches or onto only the corresponding stroma (i.e., LFs or MSCs) after an additional 10 days (FIG. 2a). Whereas T4-2 cells grew extensively on lung and BoMa stroma, growth of T4-2 cells on organotypic microvascular niches was reduced drastically (3-fold in lung-like- and 5-fold in BoMa-like-microenvironments; FIGS. 2B, 2C, and 2D). Similar results were obtained also with a luminal, estrogen receptor-positive (ER⁺) BCC line (MCF-7) (FIGS. 9a and 9b). Highly metastatic MDA-MB-231 cells displayed the same trend; in particular, cells adherent to microvasculature were Ki67-negative (FIGS. 9C, 9D, and 9E). Ki67 immunofluorescence (FIG. 2B, inset) revealed further that the vast majority of T4-2 cells seeded in organotypic microvascular niches became quiescent (77.4% Ki67-negative clusters in lung-like niche and 88.1% in BoMa-like niche; FIGS. 2E and 2F). Importantly, this was not a transient phenotype, as microvasculature-associated tumor clusters remained dormant by and large, as opposed to BCCs cultured only on lung or BoMa stroma (FIGS. 2G, 2H, 2I, and 2J). Thus, our organotypic models recapitulated our in vivo findings and allowed us to pinpoint ECs as a prime regulator of DTC quiescence in lung and BoMa. We next sought to identify endothelium-derived factor(s) underlying this effect.

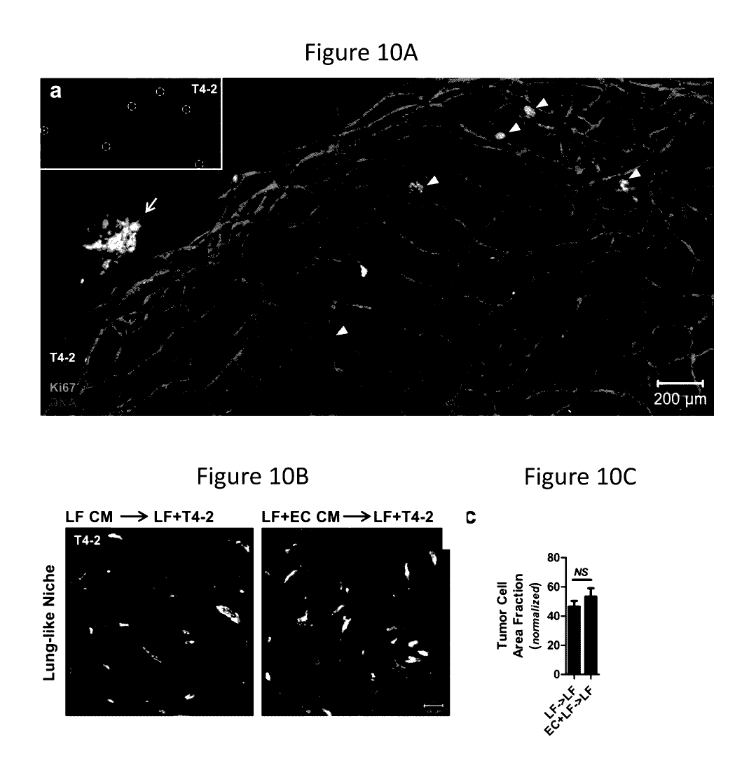

We noted consistently that whereas the bulk of quiescent tumor clusters remained on or near microvascular endothelium in our culture models, those that had seeded—or strayed—to the edge of a well and off of microvasculature typically underwent drastic expansion (FIG. 10a; inset shows that all clusters originated from single cells). This observation hinted that the putative angiocrine tumor suppressor(s) was not freely diffusible. Indeed, medium conditioned daily by microvascular niche cultures did not reduce T4-2 cell growth on lung stroma when compared to control conditions (FIGS. 10B and 10C). Accordingly, to identify factors deposited locally by ECs that could suppress tumor cell growth, we performed comparative proteomics on decellularized extracellular matrix (ECM) from lung- and BoMa-like microvascular niches (versus their respective stroma). A number of extracellular factors were upregulated in organotypic microvascular niches (FIG. 3A). Among these potential angiocrine tumor suppressors, TSP-1 caught our attention because: i) TSP-1 was expressed at higher levels in both organotypic lung- and BoMa-microvascular niches compared to stroma alone (FIG. 3a), and ii) TSP-1 overexpression in BCCs was shown previously to suppress metastatic outgrowth in lung (Weinstat-Saslow, D. L., et al. Transfection of thrombospondin 1 complementary DNA into a human breast carcinoma cell line reduces primary tumor growth, metastatic potential, and angiogenesis. Cancer Res 54, 6504-6511 (1994)). However, these anti-tumor effects were attributed to the anti-angiogenic activity of TSP-1. The possibility that TSP-1 could function to directly suppress tumor cell growth (particularly from a non-tumor source within the DTC microenvironment) had not been considered (Roberts, D. D. Regulation of tumor growth and metastasis by thrombospondin-1. FASEB J 10, 1183-1191 (1996)).

We verified first that TSP-1 was present on lung microvessels associated with dormant DTCs in both spontaneous and experimental metastasis models (FIGS. 3B and 3C). We confirmed also that TSP-1 is expressed in non-tumor bearing mice in the microvascular BM of murine lung (FIG. 3D), bone (FIG. 3E) and brain (FIG. 3F). Similar peri-endothelial localization was observed also in organotypic microvascular niches. To determine whether perivascular TSP-1 is derived primarily from ECs, we utilized a 3D co-culture model consisting of EC-coated microcarrier beads embedded within a fibrin ECM several millimeters away from overlaid LFs. ECs form robust microvascular networks after 7 days under these conditions (Ghajar, C. M., et al. The effect of matrix density on the regulation of 3-D capillary morphogenesis. Biophys J 94, 1930-1941 (2008)), and TSP-1 was concentrated within the BM of established microvessels (FIGS. 3G and 3H). Gain-of-function studies confirmed that TSP-1 was sufficient to suppress BCC growth on lung stroma in the absence of endothelium (FIG. 3I). Additionally, pre-treatment with a TSP-1 blocking antibody to interfere with T4-2 cell adhesion to TSP-1 within lung-like microvascular niches resulted in significantly increased tumor cell outgrowth compared to IgG control-treated cultures (FIG. 3J). These gain- and loss-of function experiments, combined with its presence in microvascular BM and its association with dormant DTCs, identified TSP-1 as an angiocrine tumor suppressor.

### Neovascular Tips Accelerate Breast Tumor Cell Outgrowth

Because TSP-1 stabilizes microvascular endothelium by inhibiting EC motility and growth (Roberts, D. D. Regulation of tumor growth and metastasis by thrombospondin-1. FASEB J 10, 1183-1191 (1996)), it was not surprising to find it expressed surrounding established microvasculature (FIGS. 3D, 3E, 3F and 3G). However, loss of TSP-1 expression at neovascular tips (FIGS. 3G 3H) suggested that this physiological ‘knockdown’ could result in a concomitant loss of tumor suppression within neovascular sub-niches. In support of this idea, we found that quiescent tumor clusters were often associated with stable endothelial stalks (FIG. 4A(i); asterisk, and also FIGS. 4A(ii), 4A(iii), and 4A(iv), all from the same culture), whereas actively growing tumor clusters were often surrounded by sprouting neovascular tips (FIG. 4A(i); ‘T’). Therefore, we hypothesized that these two sub-niches exert differential growth control over BCCs.

Malignant T4-2 cells expressing histone H2B-GFP were seeded on top of microvascular niches and tracked for 72 h. Qualitative analysis of time-lapse videos revealed that tumor cells remaining near established vessel stalks divided more slowly than those that encountered neovascular tips (FIG. 4B). To perform quantitative analysis, we defined 3 sub-niches: ‘neovascular tip’ for tumor cells within 50 μm of a sprouting endothelial tip, ‘stable endothelium’ for tumor cells within 50 μm of established, non-invasive endothelium and ‘stroma’ for tumor cells >50 μm away from either type of endothelium. We quantified the aggregate time (i.e., dwell time, t_{dwell}) that 229 tumor cells spent in each of these sub-niches well, before undergoing a single division (division time: t_{div}). Thus, in FIG. 4C, the scatter plot represents the fraction of each T4-2 cell's t_{div} spent near stable (red) or neovascular (green) endothelium, or on stroma (black). Pearson correlation analysis revealed that dwell time around stable endothelium (t_{dwell, stable}) correlated significantly with t_{div} (two-tailed p value=0.014); i.e., BCCs with longer division times tended to reside longer near established endothelium (FIG. 4c, red trend line). Conversely, tumor cell dwell time around neovascular tips (t_{dwell, neo}) anti-correlated significantly with t_{div} (two-tailed p value=0.001; green trend line in FIG. 4C). Importantly, stromal dwell time (t_{dwell, stroma}) did not correlate with t_{div} (FIG. 4C, black trend line) at all. We extended this analysis further by examining the fastest— (t_{div}<t_{avg}−SD) and slowest—(t_{div}>t_{avg}+SD) dividing tumor cells and found that the fastest dividing tumor cells resided 2.1-times longer within neovascular sub-niches than around stable endothelium, whereas the slowest dividing tumor cells did the opposite (FIG. 4D).

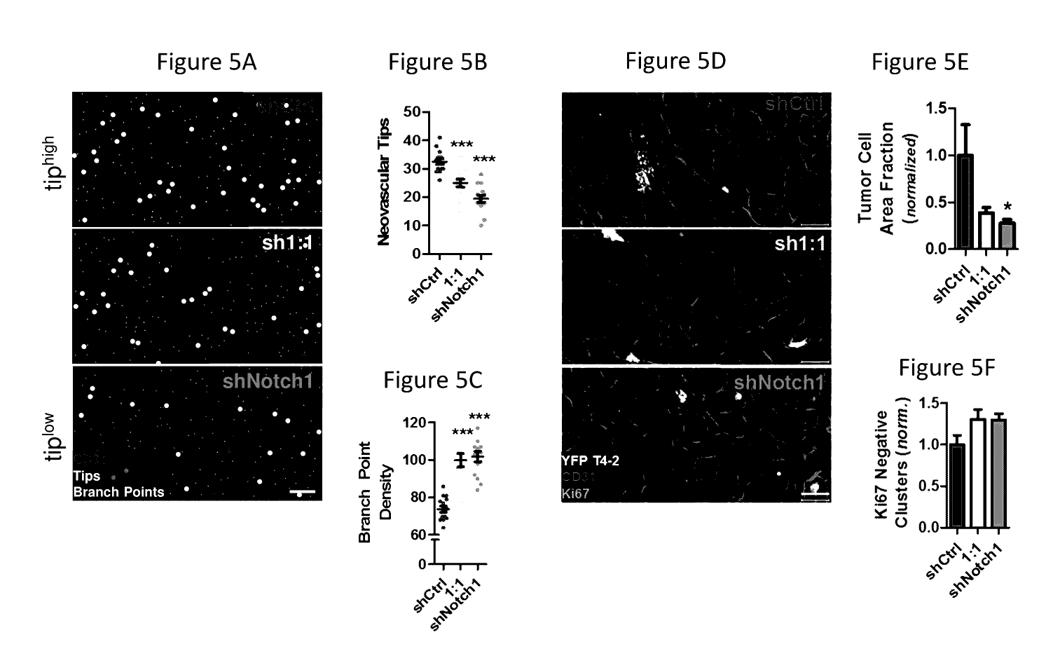

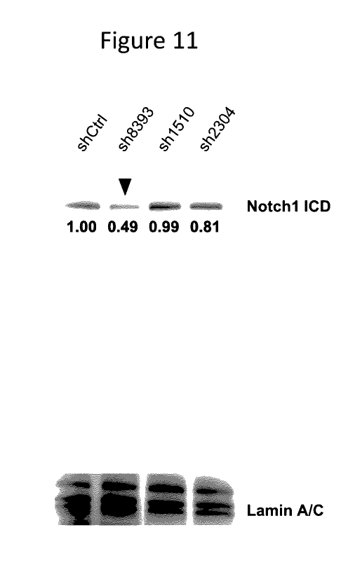

Our analysis suggested that established endothelium steers BCCs towards a quiescent phenotype, whereas neovascular endothelium accelerates BCC growth. If this were indeed true, tumor growth should decrease if neovascular tips are depleted prior to tumor cell seeding, and increase if neovascular tip formation is promoted. Consistent with observations from the developing mouse retina (Hellstrom, M., et al. D114 signalling through Notch1 regulates formation of tip cells during angiogenesis. Nature 445, 776-780 (2007)), reduced expression of endothelial Notch1 via Notch-1 targeting shRNA (FIG. 11) led to a hyperbranched network, but with a significant reduction in the number of endothelial tips (FIGS. 5A, 5B, and 5C). This was to be expected from a ‘closed system’ (our case), as opposed to an ‘open system’ (the developing retina³⁰). BCC growth followed suit, evidenced by the progressive reduction in growth of T4-2 cells seeded on either shCtrl (tip^{high})-, shCtrl:shNotch1 chimera (sh1:1)- or shNotch1 (tip^{low})-EC cultures (FIGS. 5D and 5E). The percentage of quiescent tumor clusters also increased modestly in cultures that contained fewer neovascular tips (FIG. 5F).

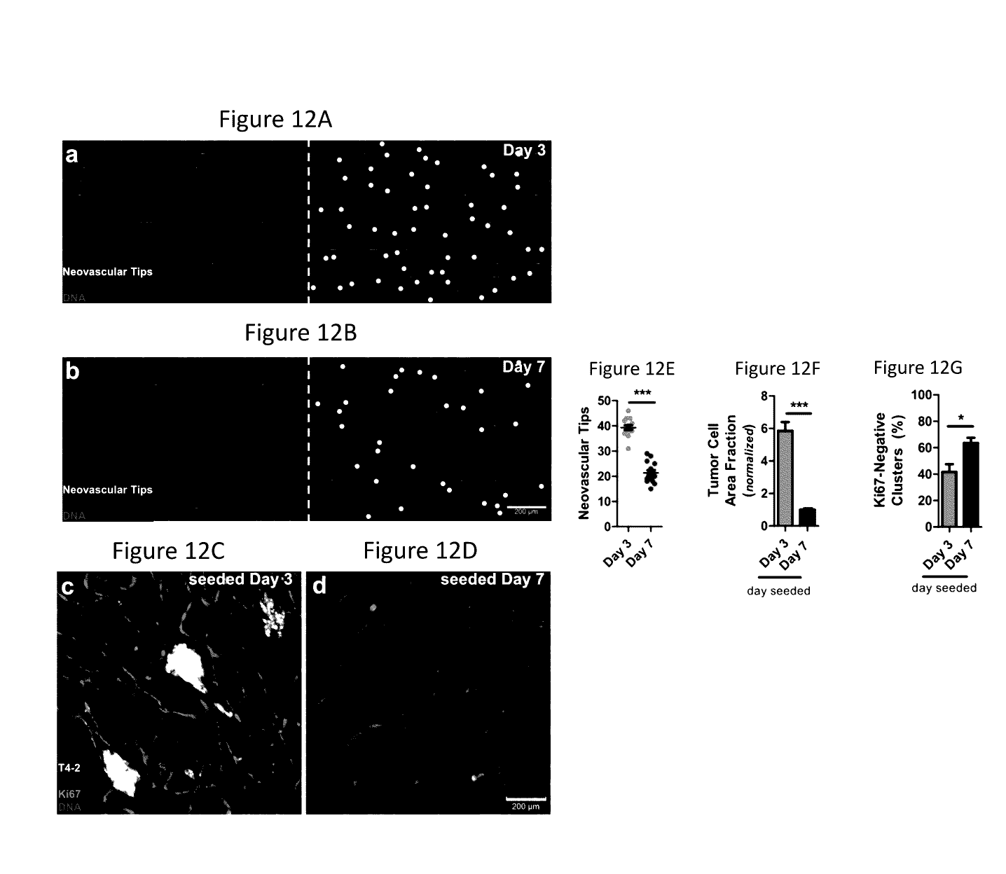

We tested also whether increased neovascular tip concentration would promote tumor cell growth in culture and in vivo. To enrich for neovascular tips in culture, we allowed microvascular networks to develop for only 3 days prior to seeding T4-2 cells. The number of neovascular tips at day 3 of network formation was nearly double that of day 7 cultures (FIGS. 12A, 12B, and 12E). Seeding tumor cells at each of these developmental time points and measuring growth 10 days later confirmed that BCC growth correlates positively with endothelial tip number; T4-2 cells grew nearly 6-times more when seeded on networks rich in neovascular tips, and significantly fewer of these tumor clusters became quiescent (FIGS. 12C, 12D, 12F, and 12G).

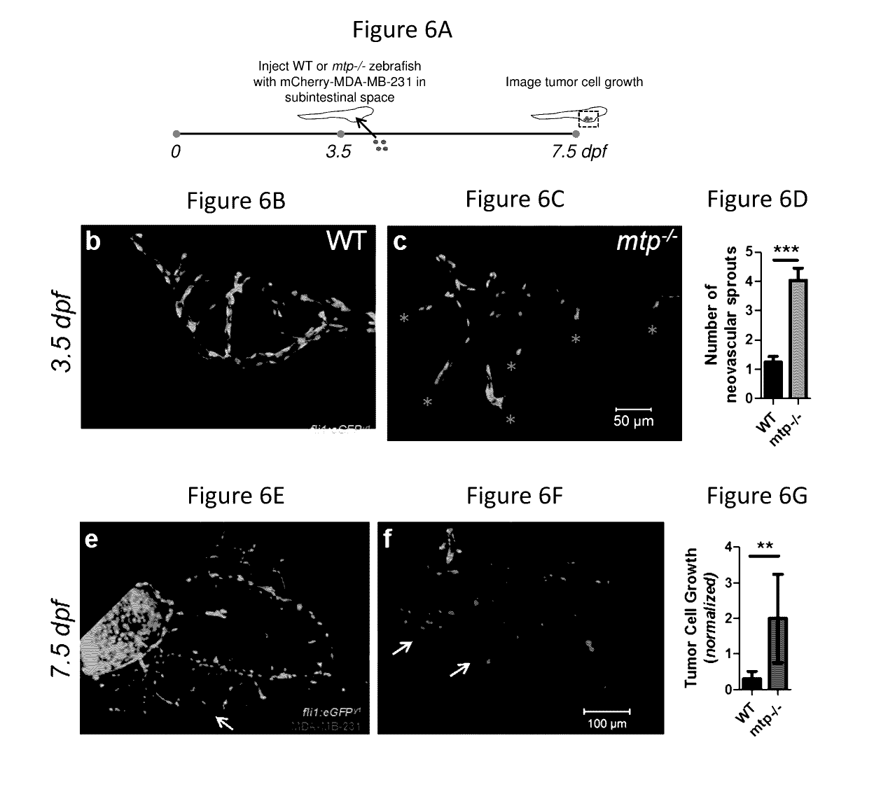

To test whether a microenvironment rich in neovascular tips promotes tumor cell growth in vivo, we utilized zebrafish with a mutation in the gene encoding microsomal triglyceride transfer protein (mtp). These mutants, called stalactite, have an ectopic microvascular sprouting phenotype that is especially pronounced in the perivitelline/subintestinal space at 3.5 days post-fertilization (dpf) (Avraham-Davidi, I., et al. ApoB-containing lipoproteins regulate angiogenesis by modulating expression of VEGF receptor 1. Nat Med 18, 967-973 (2012)). Accordingly, we injected ˜1-10 MDA-MB-231 cells expressing mCherry into the subintestinal space of wild-type (WT) and mtp−/− mutant zebrafish at this timepoint. Fish injected unsuccessfully, defined as those lacking red fluorescence in the subintestinal space, or those over-injected (an area fraction of red fluorescence over a pre-determined threshold value), were discounted from further analysis. Successfully injected zebrafish were imaged four days later (7.5 dpf; mtp−/− mutants perish shortly thereafter (See also Avraham-Davidi, I., et al. ApoB-containing lipoproteins regulate angiogenesis by modulating expression of VEGF receptor 1. Nat Med 18, 967-973 (2012))—FIG. 6A). On average, mtp−/− mutants had 4-times more neovascular sprouts than their WT siblings at the time of injection (FIGS. 6B, 6C, and 6D). In WT fish that survived until 7.5 dpf and had viable MDA-MB-231 cells in their subintestinal space, those that adhered to subintestinal vessels did not grow appreciably (FIGS. 6E and 6G; note that for each fish, tumor cell area fraction at 7.5 dpf was normalized to the corresponding value obtained just after injection to account for variations in initial seeding density). In contrast, tumor cells injected into the subintestinal space of mtp−/− mutants expanded significantly more than those in WT siblings, particularly in the vicinity of neovascular tips (FIGS. 6F and 6G).

### Neovascular Tips Constitute ‘Micrometastatic Niches’ Rich in Periostin and Active TGF-β1

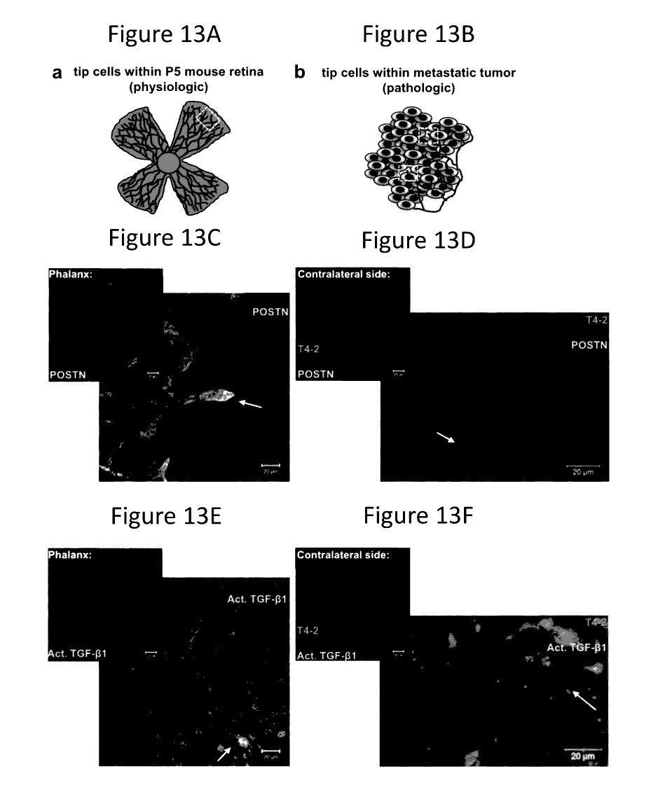

The above experiments confirmed that neovascular tips promote tumor cell outgrowth in organotypic culture and in vivo, implying production of distinct tumor-promoting factors by neovascular tip cells. To identify factors enriched around neovascular tips, we utilized tandem mass spectrometry and compared decellularized ECM from neovascular tip^{high} (shCtrl) and tip^{low} (shNotch1) cultures (FIG. 5A). Tip^{high} cultures were characterized by enhanced expression of POSTN, tenascin, versican, and fibronectin (FIG. 7A), all molecules involved in formation of the metastatic niche (Kaplan, R. N., et al. VEGFR1-positive haematopoietic bone marrow progenitors initiate the pre-metastatic niche. Nature 438, 820-827 (2005); Kim, S., et al. Carcinoma-produced factors activate myeloid cells through TLR2 to stimulate metastasis. Nature 457, 102-106 (2009); Malanchi, I., et al. Interactions between cancer stem cells and their niche govern metastatic colonization. Nature 481, 85-89 (2012); Oskarsson, T., et al. Breast cancer cells produce tenascin C as a metastatic niche component to colonize the lungs. Nat Med 17, 867-874 (2011); Soikkeli, J., et al. Metastatic outgrowth encompasses COL-I, FN1, and POSTN up-regulation and assembly to fibrillar networks regulating cell adhesion, migration, and growth. Am J Pathol 177, 387-403 (2010)). Further, tip^{high} cultures exhibited reduced expression of molecules involved in sequestering another known mediator of metastatic outgrowth, TGF-β1 (biglycan and LTBP1, FIG. 7A), suggesting that active TGF-β1 itself would be expressed more highly at neovascular tips. Immunofluorescent staining of E4-ECs in 3D co-cultures confirmed that active TGF-β1 and POSTN were expressed highly at neovascular tips (FIGS. 7B, 7C, and 7E). In contrast, latent TGF-β1 was expressed prominently in endothelial stalks (FIG. 7d). These findings were confirmed in vivo in physiologic and pathologic settings. POSTN and active TGF-β1 were concentrated on/near endothelial tip cells in the developing mouse retina (FIGS. 13A, 13C, and 13E), but were not detected consistently around established ‘phalanx’ endothelium in the same tissue (FIGS. 13C and 13E; insets). Examining established metastases revealed that in this setting, POSTN and active TGF-β1 were deposited prominently around endothelial tip cells as well (FIGS. 13B, 13D, and 13F). In contrast, expression of both factors near non-tumor-associated endothelium within the same tissue sections was diminished (FIGS. 13D and 13F; insets). Pulsing POSTN and TGF-β1 into microvascular niche cultures to recapitulate a tip-enriched microenvironment promoted BCC growth; T4-2 cells seeded upon lung-like microvascular niches and treated transiently with POSTN and TGF-β1 (twice over the first 48 h to mimic the brief interaction of tumor cells with neovascular tips) experienced 3-times more outgrowth when compared to vehicle treated counterparts (FIGS. 7F, 7G and 7H). This finding confirms that POSTN and TGF-β1, which are expressed highly at neovascular tips, promote BCC outgrowth within a tumor suppressive microenvironment.

Discussion.

Using murine models, zebrafish and organotypic microvascular niches composed of human cells, we demonstrate here that: i) dormant DTCs from the breast reside on or near lung and BoMa microvasculature in vivo, ii) stable microvasculature constitutes a dormant niche that induces sustained tumor cell quiescence via TSP-1, and iii) the tumor-suppressive nature of microvascular endothelium is lost at sprouting endothelial tips, which are characterized by reduced TSP-1 expression and enhanced expression of pro-tumor factors POSTN and TGF-β1 (FIG. 7I). Studies of primary tumors have focused primarily on the tumor's regulation of the endothelium. We believe this paradigm now shifts at secondary sites, where DTCs are the minority constituent of the tissue and subject to direct control by microvascular endothelium and perhaps other resident cell types.

The notion that ECs directly regulate cells in the perivascular microenvironment is rooted in a number of biological studies on normal tissues (reviewed in Butler, J. M., Kobayashi, H. & Rafii, S. Instructive role of the vascular niche in promoting tumour growth and tissue repair by angiocrine factors. Nat Rev Cancer 10, 138-146 (2010)), ECs with phenotypic characteristics of neovascular tip cells spark growth and morphogenesis of the liver (Matsumoto, K., Yoshitomi, H., Rossant, J. & Zaret, K. S. Liver organogenesis promoted by endothelial cells prior to vascular function. Science 294, 559-563 (2001)) and regeneration of lung alveoli (Ding, B. S., et al. Endothelial-derived angiocrine signals induce and sustain regenerative lung alveolarization. Cell 147, 539-553 (2011)). On the other hand, established endothelium promotes pancreatic differentiation (Lammert, E., Cleaver, O. & Melton, D. Induction of pancreatic differentiation by signals from blood vessels. Science 294, 564-567 (2001)), inhibits smooth muscle cell proliferation (Dodge, A. B., Lu, X. & D'Amore, P. A. Density-dependent endothelial cell production of an inhibitor of smooth muscle cell growth. J Cell Biochem 53, 21-31 (1993)) and maintains pluripotency of neural, hematopoietic and mesenchymal stem cells (See Butler, J. M., et al. Endothelial cells are essential for the self-renewal and repopulation of Notch-dependent hematopoietic stem cells. Cell Stem Cell 6, 251-264 (2010); Crisan, M., et al. A perivascular origin for mesenchymal stem cells in multiple human organs. Cell Stem Cell 3, 301-313 (2008), Kobayashi, H., et al. Angiocrine factors from Akt-activated endothelial cells balance self-renewal and differentiation of haematopoietic stem cells. Nat Cell Biol 12, 1046-1056 (2010); Shen, Q., et al. Endothelial cells stimulate self-renewal and expand neurogenesis of neural stem cells. Science 304, 1338-1340 (2004); and Ding, L., Saunders, T. L., Enikolopov, G. & Morrison, S. J. Endothelial and perivascular cells maintain haematopoietic stem cells. Nature 481, 457-462 (2012)). Our study demonstrates that this scenario—that mature microvasculature confers tissue quiescence and sprouting endothelium promotes tissue growth—is at work also in the DTC microenvironment. These findings may apply generally to primary tumors also, thus shedding light on the apparent dichotomy of EC function at the primary site⁴⁵⁻⁴⁹.

The therapeutic implications of our results are multi-fold. Foremost is that identification of tumor-suppressive factors derived from stable endothelium may guide therapies designed to enforce DTC dormancy. This raises the question of whether other molecules in the microvascular BM function as tumor suppressors, and whether these can be used in combination with TSP-1 to stave off metastatic relapse. Second is that factors enriched in neovascular sub-niches may be targeted early in tumor progression to prevent establishment of micro-metastatic niches that disrupt DTC quiescence. In this regard, our study complements prior work pinpointing POSTN, TGF-β1 and other molecules as potential therapeutic targets (See Kim, S., et al. Carcinoma-produced factors activate myeloid cells through TLR2 to stimulate metastasis. Nature 457, 102-106 (2009); Malanchi, I., et al. Interactions between cancer stem cells and their niche govern metastatic colonization. Nature 481, 85-89 (2012); Oskarsson, T., et al. Breast cancer cells produce tenascin C as a metastatic niche component to colonize the lungs. Nat Med 17, 867-874 (2011); Soikkeli, J., et al. Metastatic outgrowth encompasses COL-I, FN1, and POSTN up-regulation and assembly to fibrillar networks regulating cell adhesion, migration, and growth. Am J Pathol 177, 387-403 (2010); Bierie, B. & Moses, H. L. Tumour microenvironment: TGFbeta: the molecular Jekyll and Hyde of cancer. Nat Rev Cancer 6, 506-520 (2006)), and reveals further that these molecules arise from an unexpected source, namely neovascular endothelium.

Surprisingly, many of the factors upregulated in neovascular tip-enriched cultures are documented components of pre-metastatic and metastatic niches (See references above). Given the nature of our results, this provides further evidence for the in vivo relevance of our model systems, but also raises a number of questions regarding the origin of this commonality, and whether the commonality reflects a tight interconnectedness of metastatic niche formation on the induction of neovasculature. It is interesting to note that nascent endothelium was recently shown to initiate a Th2-mediated inflammatory response in asthma (Asosingh, K., et al. Nascent endothelium initiates th2 polarization of asthma. J Immunol 190, 3458-3465 (2013)), a response that is also associated with accelerated metastatic outgrowth in tumor models (Lin, E. Y., Nguyen, A. V., Russell, R. G. & Pollard, J. W. Colony-stimulating factor 1 promotes progression of mammary tumors to malignancy. J Exp Med 193, 727-740 (2001); Qian, B. Z. & Pollard, J. W. Macrophage diversity enhances tumor progression and metastasis. Cell 141, 39-51 (2010); DeNardo, D. G., et al. CD4(+) T cells regulate pulmonary metastasis of mammary carcinomas by enhancing protumor properties of macrophages. Cancer Cell 16, 91-102 (2009)). Thus, by direct deposition of tumor promoting factors, as well as by secreting cytokines that stimulate macrophage polarization to a pro-tumor phenotype, neovascular tips may function as a nexus that directly and indirectly catalyzes formation of a micrometastatic niche. Accordingly, long-term administration of drugs aimed at preventing neovascular formation (Folkman, J. Angiogenesis: an organizing principle for drug discovery? Nat Rev Drug Discov 6, 273-286 (2007)) through inhibition of VEGFR2— (Jakobsson, L., et al. Endothelial cells dynamically compete for the tip cell position during angiogenic sprouting. Nat Cell Biol 12, 943-953 (2010).) or integrin αᵥβ₃-(Brooks, P. C., Clark, R. A. & Cheresh, D. A. Requirement of vascular integrin alpha v beta 3 for angiogenesis. Science 264, 569-571 (1994)) driven signaling, or by targeting more recently discovered pro-angiogenic signaling mechanisms (Stratman, A. N., Davis, M. J. & Davis, G. E. VEGF and FGF prime vascular tube morphogenesis and sprouting directed by hematopoietic stem cell cytokines. Blood 117, 3709-3719 (2011)), may prove effective in delaying relapse of early stage breast cancer patients. We believe that it will be crucial to deliver these drugs in a manner that prevents cultivation of the pro-tumor neovascular niche while preserving the dormant niche fostered by stable microvasculature.

It remains to be determined whether the mechanisms we have identified here apply also to other tumor types and in other secondary tissues. We propose that a systemic understanding of interactions between DTCs and their microenvironment will provide a vehicle by which we can design more effective therapies to keep DTCs at bay—or eradicate them—in early-stage cancer patients.

### Materials and Methods

### Animal Studies.

Metastasis Assays.

All mouse work was performed in accordance with institutional, IACUC and AAALAS guidelines. For spontaneous metastasis assays, GFP-luc MDA-MB-231 (1×10⁶ cells) were injected into the inguinal mammary gland of 7-wk-old female NOD-SCID mice (20 total; Charles River) in a 1:1 solution of LrECM (Growth-factor reduced Cultrex; Trevigen): Dulbecco's Modified Eagle's Medium (DMEM; Invitrogen/Gibco). Tumors 0.5 cm³ were resected 3 wks later. Mice were monitored weekly for relapse by BLI and those that did not experience gross metastatic relapse early on were sacrificed and dissected at 6 wks. Lungs were harvested after saline perfusion. Primary tumors and lungs were fixed overnight in 1.6% paraformaldehyde (PFA)/PBS solution and then banked in optimum cutting temperature (OCT) compound (Tissue-Tek). Femurs and tibia were fixed in identical fashion and then decalcified by gentle shaking in decalcification solution (0.1M Tris-HCl, 0.26M EDTA, pH=7.4) for 1 wk protected from light and with intermittent changes of decalcification solution before overnight (O/N) incubation in 30% sucrose, incubation in 1:1 sucrose:OCT (1 h-O/N), and finally embedding in OCT compound.

For experimental metastasis assays, mCherry-T4-2 cells (1×10⁵ cells in 100 μl PBS) were injected into the left cardiac ventricle of 6-8 wk old female NOD-SCID mice with a 26½ gauge needle. Successful injection was characterized by the pumping of arterial blood into the syringe. Mice that did show any signs of tumor burden were sacrificed and dissected 8 wks post-injection. Tissues were processed as described above.

Retinal Angiogenesis Assay.

Retinas were dissected from P5 C57BL/6 mice, whole-mounted and stained as described in Pitulescu, M. E., Schmidt, I., Benedito, R. & Adams, R. H. Inducible gene targeting in the neonatal vasculature and analysis of retinal angiogenesis in mice. Nat Protoc 5, 1518-1534 (2010) hereby incorporated by reference. Antibodies used for staining are detailed below.

Zebrafish Xenografts.

Establishment and characterization of Tg(fli1:eGFP)^{y1} and mtp−/− (a.k.a. stalactite) mutant lines have been described in Avraham-Davidi, I., et al. ApoB-containing lipoproteins regulate angiogenesis by modulating expression of VEGF receptor 1. Nat Med 18, 967-973 (2012) and were generously provided by Brant Weinstein (NICHD/NIH). Embryos and adults were maintained under standard laboratory conditions, as described previously (Stratman, A. N., Davis, M. J. & Davis, G. E. VEGF and FGF prime vascular tube morphogenesis and sprouting directed by hematopoietic stem cell cytokines. Blood 117, 3709-3719 (2011), hereby incorporated by reference). Injection of mCherry-MDA-MB-231 cells into the perivitelline/subintestinal space of 3.5 dpf mtp−/− mutants and WT siblings was conducted essentially as described (Nicoli, S. & Presta, M. The zebrafish/tumor xenograft angiogenesis assay. Nat Protoc 2, 2918-2923 (2007) hereby incorporated by reference), except the cellular solution was diluted such that ˜1-10 MDA-MB-231 cells were injected per fish. Fish were incubated for four days post-injection and fixed at 7.5 dpf. Quantification was performed as detailed below.

Immunofluorescent Staining.

Serial tissue sections (thickness: 50 μm) of primary tumors, lungs, bones, and brains were generated with a Leica Cryostat CM3050 S (Leica Microsystems). Sections were thawed, rehydrated in PBS and incubated in 0.1M Glycine/PBS 0/N to neutralize PFA activity. Tissues were then rinsed extensively with PBS and stained as described in Baluk, P., Morikawa, S., Haskell, A., Mancuso, M. & McDonald, D. M. Abnormalities of basement membrane on blood vessels and endothelial sprouts in tumors. Am J Pathol 163, 1801-1815 (2003) hereby incorporated by reference. In tissue sections and in whole-mounted retina, endothelial cells were labeled with a rat monoclonal antibody targeting CD31/PECAM-1(BD Pharmingen 553373, clone: MEC 13.3, 1:250), TSP-1 was stained with a rabbit polyclonal antibody (AbCam ab85762, 5 μg/ml), POSTN was stained with a mouse monoclonal antibody (AdipoGen AG-20B-0033, clone: Stiny-1; 5 μg/ml), active TGF-β1 was stained with a chicken polyclonal antibody (R&D Systems AF-101-NA, 2 μg/ml), and proliferating cells were identified with a rabbit polyclonal antibody targeting Ki67 (Vector Laboratories VP-K451, 1:500) or a mouse monoclonal antibody targeting PCNA (Abcam ab29, clone: PC10, 1 μg/ml). Hoechst 33342 (Sigma) was used to label cellular nuclei. Secondary antibodies used were goat anti-rat 488 or 568 and goat anti-rabbit 405 or 633 (Invitrogen), all at 1:500. Tissues were imaged on a Zeiss LSM 710 confocal microscope using either a 1.1NA 40× water-immersion objective or a 1.4NA 63× oil-immersion objective.

3D cultures (see below) were stained after fixation with Alexa fluor 568 Phalloidin (Invitrogen A12380, 1:200) to detect F-actin or with the following antibodies: mouse monoclonal antibody targeting human CD31/PECAM-1 (Millipore CBL468, clone: HC1/6 1:200), rabbit polyclonal antibody to Ki67 (see above), rabbit polyclonal antibody to periostin (AbCam ab14041, 1:100), chicken polyclonal antibody to active TGF-β1 (see above), goat polyclonal antibody to LAP TGF-β1 (R&D Systems AB-246-BA, 10 μg/ml), and mouse monoclonal antibody to type IV collagen (University of Iowa Developmental Studies Hybridoma Bank, clone: M3F7, 1:100).

Cell Culture and Reagents.

HUVEC isolated freshly from human umbilical cord veins were propagated in EGM-2 growth medium (Lonza). Human MSCs and LFs were obtained commercially (Lonza) and propagated in low glucose (MSCs) or high glucose (LFs) DMEM supplemented with 10% fetal bovine serum (FBS, Atlanta Biologicals) and 1% penicillin/streptomycin (P/S; UCSF Cell Culture Facility). All primary human cells were used in experiments before passage 10.

Malignant T4-2 cells were grown in H14 medium on collagen-coated tissue culture flasks. MCF-7 and MDA-MB-231 cells were grown in high glucose DMEM supplemented with 10% FBS and 1% P/S.

mCherry-E4-ECs were generated by retroviral infection of E4-ECs with a pBMN/mCherry plasmid as described in Ghajar, C. M., et al. The effect of matrix density on the regulation of 3-D capillary morphogenesis. Biophys J 94, 1930-1941 (2008), hereby incorporated by reference in its entirety. YFP-T4-2, -MCF-7, and -MDA-MB-231 were generated by infection of tumor cells with pLentiCMV/YFP lentivirus followed by selection for 96 h in 1 μg/ml puromycin. Histone H2B-GFP T4-2 have been described previously in Tanner, K., Mori, H., Mroue, R., Bruni-Cardoso, A. & Bissell, M. J. Coherent angular motion in the establishment of multicellular architecture of glandular tissues. Proc Natl Acad Sci USA (2012), hereby incorporated by reference in its entirety.

Generation of E4ORF1 Lentivirus and E4ORF1-HUVEC.

pCCL-PGK lentiviral vector containing the human adenoviral E4ORF1 gene (serotype 5) was a kind gift from Shahin Rafii (Weill Cornell Medical College, HHMI) and described in Seandel, M., et al. Generation of a functional and durable vascular niche by the adenoviral E4ORF1 gene. Proc Natl Acad Sci USA 105, 19288-19293 (2008), hereby incorporated by reference. Lentivirus was generated by co-transfection of sub-confluent 293 FT cells with 2 μg each of PLP1, PLP2, VSVG and E4ORF1 plasmid DNA in DMEM containing a 3:1 (μl:μg) ratio of FuGene6 (Roche):total plasmid DNA. 293FT medium was changed to growth medium 24 h after transfection and lentivirus was collected 48 h later. HUVEC were infected at a multiplicity of infection (MOI) of 5 using Mission ExpressMag Supermagnetic Kit (Sigma) per manufacturer's instructions, then ‘selected’ for 96 h in totally unsupplemented DMEM/F12 medium.

Microvascular Niche Cultures.

Microvascular niche cultures were generated with modifications to a previously described protocol, described in Evensen, L., et al. Mural cell associated VEGF is required for organotypic vessel formation. PLoS One 4, e5798 (2009), hereby incorporated by reference in its entirety. LFs or MSCs were seeded alone at a density of 5×10⁴ cells/well in 96-well culture plates or with mCherry-E4-ECs at a 5:1 ratio to generate lung-like or BoMa-like microvascular niches, respectively. Cells were suspended in EGM-2 at a concentration 5×10⁴ cells/100 μl (stroma only) or 6×10⁴ cells/100 μl (stroma+ECs). After depositing 100 μl of cellular suspension per well of a 96-well plate, plates were left undisturbed on a flat surface for 20 min to allow even cell seeding prior to incubation.

After 7 days, YFP tumor cells were suspended in unsupplemented DMEM/F12 (800 cells/ml). YFP tumor cells were seeded (100 μl/well) after washing cultures thrice with PBS. Cells were allowed to settle for 15 min at room temperature, then a “drip” of LrECM⁶⁴ in DMEM/F12 was slowly added to each well (final concentration=20%). Drip condensed for 10 min at room temperature before polymerizing fully at 37° C. prior to imaging. Cultures were imaged immediately after seeding on a Zeiss LSM 710 confocal microscope using a 0.3 NA 10× air objective. The objective was centered to each well before acquisition of 6×6 tiles that captured the near-entirety of each well. Cultures were maintained with media changes every 72 h and imaged again at day 10.

For TSP-1 blocking antibody experiments, cultures were treated at day 5 and again at day 7 (upon tumor cell seeding) with 20 μg/ml of a mouse monoclonal antibody that blocks binding of CD47 to TSP-1 (Thermoscientific MS-420-P1ABX, clone: C6.7), or with 20 μg/ml of IgG₁ control (Acris Antibodies AM03095AF-N).

Time-Lapse Acquisition.

Time-lapse sequences were acquired with a Zeiss LSM 710 confocal microscope fitted with an environmental chamber to maintain temperature (37° C.), humidity and CO₂ (5%). H2B-GFP T4-2 cells were “starved” for 24 h in unsupplemented DMEM/F12 prior to seeding on microvascular niches (see above). Images (6×6 tiles, 512×512 resolution, 8-bit) were acquired every 20 min for 72 h. Medium was replenished at 24 h.

3D Sprouting Angiogenesis Assay.

E4-EC were coated on dextran microcarrier beads (Sigma), suspended within a 3 mg/ml solution of bovine fibrinogen (Sigma), and gelled within a No 1.5 thickness 8-well borosilicate chamber slide (Thermo Scientific/Nunc) using 50 U/mL (1:25 v:v) thrombin (Sigma). 2×10⁴ LFs were overlaid in 250 μl of EGM2 per well. Cultures were analyzed at day 7.

### Quantification.

Normalized Tumor Cell Area Fraction:

A macro was written using NIH ImageJ open source software to remove bias from data quantification. For YFP channel only, day 0 images (i.e., just after tumor cell seeding) were subjected to the following: contrast was enhanced such that 0.5% of pixels were saturated. The image was then sharpened and the “Find Edges” function was applied to further enhance contrast between YFP cells and background. A constant threshold was then applied to all samples within a given experiment to eliminate variability. The total area fraction of the 6×6 tiled image occupied by YFP cells was then calculated. For Day 10 images, “Find Edges” function was not used because it created artifacts within larger tumor clusters. For each image, the measured area fraction at day 10 was normalized by the corresponding day 0 value in order to account for any small variations in seeding density from well-to-well.

Tumor Cell Area Fraction in Zebrafish:

Zebrafish were imaged immediately after injection (3.5 dpf) with a Zeiss Lumar fluorescence stereoscope, and imaged again post-fixation (7.5 dpf) with a Zeiss LSM 710 confocal microscope. Z-stacks were acquired at the latter timepoint to image tumor cells throughout the subintestinal space. Only zebrafish that survived to 7.5 dpf with viable mCherry-MDA-MB-231 cells in their subintestinal space were quantified. Tumor cell area fractions were measured only for the subintestinal space at 3.5 dpf and 7.5 dpf using the macro described above. Tumor cell area fractions measured at 7.5 dpf were normalized by the corresponding values obtained post-injection to yield ‘normalized tumor cell growth’ for each animal.

Ki67-Negativity:

Tumor clusters totally devoid of nuclear Ki67 were counted manually. The Ki67 negative fraction was obtained by dividing this number by the total number of YFP clusters per well.

Division Time vs. Sub-Niche:

A 50 μm×50 μm grid was superimposed on image sequences loaded into Imaris software to facilitate measurement of the distance between H2B-GFP T4-2 cells mCherry⁺E4-EC structures. When in question, distances were measured manually using the Measurement Points tool in Imaris. H2B-GFP T4-2 cells were tracked until first evidence of division, and the total time spent in an endothelial tip sub-niche (within 50 μm of a microvascular tip), in an endothelial stalk sub-niche (within 50 μm of microvasculature but not within 50 μm of a tip), or in the stromal sub-niche (>50 μm away from microvasculature) was tabulated for each of 229 cells that could be tracked accurately during the entire 72 h time period. Analysis was conducted in blinded fashion.

Tip Number and Branch Point Density:

Network properties were counted manually using the “cell counter” application in ImageJ.

IF Intensity at Endothelial Tip:

Using ImageJ, a minimum of 15 vessels from 2 separate experiments were quantified to determine the relative intensities of TSP-1, POSTN and active TGF-β1 at the endothelial tip vs. endothelial stalk. Images were contrast enhanced (saturated pixels=0.5%) before analysis. Average intensity of a ˜150 pixel-squared region of a tip cell and a stalk cell 2-cells-removed from said tip were measured. Background intensity was subtracted from the measured intensities. Tip and stalk intensities were each normalized by the average intensity obtained for all stalks and reported as normalized average intensities.

Notch1 Knockdown.

E4ORF1-HUVECs were infected at 5 MOI with custom-made lentiviruses (Sigma) containing shRNA targeting human Notch1 in a pLKO.1-puro-CMV-TagRFP vector. Empty vector was used as a control (shCtrl). Sequences for shRNA were as follows:

sh8393 (SEQ ID NO: 1):
CCGGCTTTGTTTCAGGTTCAGTATTCTCGAGAATACTGAACCTGAAACAA
AGTTTTT
sh1510 (SEQ ID NO: 2):
CCGGCGCTGCCTGGACAAGATCAATCTCGAGATTGATCTTGTCCAGGCAG
CGTTTTT
sh2304 (SEQ ID NO: 3):
CCGGCAAAGACATGACCAGTGGCTACTCGAGTAGCCACTGGTCATGTCTT
TGTTTTT

Western Blotting.

shNotch1-E4-ECs and shCtrl-E4-ECs were lysed in 2% SDS/PBS. Twenty μg of each lysate was then separated on a Tris-Glycine 4-20% gel. Notch1 was probed with a rabbit polyclonal antibody (AbCam ab27526, 1:500). The blot was stripped and re-probed with a rabbit polyclonal antibody to the nuclear membrane protein Lamin A/C, used here as a loading control (Santa Cruz Biotechnology sc-20681, 1:2000).

### LC-MS/MS.

Sample Preparation.

Cultures were established for 7 days in EGM-2, washed extensively with PBS to remove medium, and incubated in 0.1% Triton X-100/PBS (with added protease inhibitor cocktail, EMD Biosciences) for 30 min at 4° C. to de-cellularize the cultures. After washing, cultures were incubated 0/N at 4° C. in 0.5M acetic acid solution. The following day, acetic acid was collected and protein was precipitated from the acetic acid solution via TCA/DOC precipitation method. The precipitate was washed twice in acetone, dried at room temperature, and then dissolved 0/N in 5× Invitrosol LC/MS protein solubilizer (Invitrogen) under constant agitation. Invitrosol was brought to 1× with 25 mM NH₄(HCO₃) and final protein concentration was measured by A280 using a NanoDrop spectrophotometer (Thermo Scientific). Precipitates were stored at −80° C. until analysis.

Trypsin Digestion.

30 μg of protein from each experimental condition was proteolytically cleaved by modified, sequencing grade trypsin (Promega) in 50 mM NH₄(HCO₃) digestion buffer containing 1 μg trypsin and 2 mM CaCl₂ for 16 hours at 37° C. Reactions were then acidified with 90% formic acid (2% final) to stop proteolysis. Samples were centrifuged for 30 minutes at 14,000 rpm to remove insoluble material, then subjected to LC-MS/MS analysis.

Multidimensional Chromatography and Tandem Mass Spectrometry, Interpretation of MS/MS Datasets.

Methods for LC-MS/MS and tandem mass spectra analysis were conducted essentially as described previously in Beliveau, A., et al. Raf-induced MMP9 disrupts tissue architecture of human breast cells in three-dimensional culture and is necessary for tumor growth in vivo. Genes Dev 24, 2800-2811 (2010), hereby incorporated by reference. The resulting list of proteins was culled to ECM proteins and related growth factors/cytokines by referencing protein ontology in UniProt.org. Spectral counts for each condition were normalized by one another and log₂ values of said products were plotted in heatmap format using TreeView open source software.

Statistical Analysis.

Statistical analyses were conducted with GraphPad Prism 5 software. Please see figure legends for individual N- and p-values, and specific statistical test(s) employed. Unless noted otherwise, data are reported as mean±s.e.m.

All publications, patents and references cited herein are hereby incorporated by reference in their entirety.

### REFERENCES

1. Aguirre-Ghiso, J. A. Models, mechanisms and clinical evidence for cancer dormancy. Nat Rev Cancer 7, 834-846 (2007).
2. Goss, P. E. & Chambers, A. F. Does tumour dormancy offer a therapeutic target? Nat Rev Cancer 10, 871-877 (2010).
3. Klein, C. A. Parallel progression of primary tumours and metastases. Nat Rev Cancer 9, 302-312 (2009).
4. Uhr, J. W. & Pantel, K. Controversies in clinical cancer dormancy. Proc Natl Acad Sci USA 108, 12396-12400 (2011).
5. Kaplan, R. N., et al. VEGFR1-positive haematopoietic bone marrow progenitors initiate the pre-metastatic niche. Nature 438, 820-827 (2005).
6. Peinado, H., et al. Melanoma exosomes educate bone marrow progenitor cells toward a pro-metastatic phenotype through MET. Nat Med 18, 883-891 (2012).
7. Psaila, B. & Lyden, D. The metastatic niche: adapting the foreign soil. Nat Rev Cancer 9, 285-293 (2009).
8. Suzuki, M., Mose, E. S., Montel, V. & Tarin, D. Dormant cancer cells retrieved from metastasis-free organs regain tumorigenic and metastatic potency. Am J Pathol 169, 673-681 (2006).
9. Naumov, G. N., et al. Persistence of solitary mammary carcinoma cells in a secondary site: a possible contributor to dormancy. Cancer Res 62, 2162-2168 (2002).
10. Pantel, K., et al. Differential expression of proliferation-associated molecules in individual micrometastatic carcinoma cells. J Natl Cancer Inst 85, 1419-1424 (1993).
11. Bissell, M. J. & Hines, W. C. Why don't we get more cancer? A proposed role of the microenvironment in restraining cancer progression. Nat Med 17, 320-329 (2011).
12. Boudreau, N., Sympson, C. J., Werb, Z. & Bissell, M. J. Suppression of ICE and apoptosis in mammary epithelial cells by extracellular matrix. Science 267, 891-893 (1995).
13. Spencer, V. A., et al. Depletion of nuclear actin is a key mediator of quiescence in epithelial cells. J Cell Sci 124, 123-132 (2011).
14. Weaver, V. M., et al. beta4 integrin-dependent formation of polarized three-dimensional architecture confers resistance to apoptosis in normal and malignant mammary epithelium. Cancer Cell 2, 205-216 (2002).
15. Weaver, V. M., et al. Reversion of the malignant phenotype of human breast cells in three-dimensional culture and in vivo by integrin blocking antibodies. J Cell Biol 137, 231-245 (1997).
16. Bissell, M. J., Hall, H. G. & Parry, G. How does the extracellular matrix direct gene expression? J Theor Biol 99, 31-68 (1982).
17. Petersen, O. W., Ronnov-Jessen, L., Howlett, A. R. & Bissell, M. J. Interaction with basement membrane serves to rapidly distinguish growth and differentiation pattern of normal and malignant human breast epithelial cells. Proc Natl Acad Sci USA 89, 9064-9068 (1992).
18. Braun, S., et al. A pooled analysis of bone marrow micrometastasis in breast cancer. N Engl J Med 353, 793-802 (2005).
19. Chambers, A. F., Groom, A. C. & MacDonald, I. C. Dissemination and growth of cancer cells in metastatic sites. Nat Rev Cancer 2, 563-572 (2002).
20. Francia, G., Cruz-Munoz, W., Man, S., Xu, P. & Kerbel, R. S. Mouse models of advanced spontaneous metastasis for experimental therapeutics. Nat Rev Cancer 11, 135-141 (2011).
21. Paget, S. The distribution of secondary growths in cancer of the breast. 1889. Cancer Metastasis Rev 8, 98-101 (1989).
22. Briand, P., Nielsen, K. V., Madsen, M. W. & Petersen, O. W. Trisomy 7p and malignant transformation of human breast epithelial cells following epidermal growth factor withdrawal. Cancer Res 56, 2039-2044 (1996).
23. Butler, J. M., et al. Endothelial cells are essential for the self-renewal and repopulation of Notch-dependent hematopoietic stem cells. Cell Stem Cell 6, 251-264 (2010).
24. Seandel, M., et al. Generation of a functional and durable vascular niche by the adenoviral E4ORF1 gene. Proc Natl Acad Sci USA 105, 19288-19293 (2008).
25. Butler, J. M., Kobayashi, H. & Rafii, S. Instructive role of the vascular niche in promoting tumour growth and tissue repair by angiocrine factors. Nat Rev Cancer 10, 138-146 (2010).
26. Evensen, L., et al. Mural cell associated VEGF is required for organotypic vessel formation. PLoS One 4, e5798 (2009).
27. Weinstat-Saslow, D. L., et al. Transfection of thrombospondin 1 complementary DNA into a human breast carcinoma cell line reduces primary tumor growth, metastatic potential, and angiogenesis. Cancer Res 54, 6504-6511 (1994).
28. Roberts, D. D. Regulation of tumor growth and metastasis by thrombospondin-1. FASEB J 10, 1183-1191 (1996).
29. Ghajar, C. M., et al. The effect of matrix density on the regulation of 3-D capillary morphogenesis. Biophys J 94, 1930-1941 (2008).
30. Hellstrom, M., et al. D114 signalling through Notch1 regulates formation of tip cells during angiogenesis. Nature 445, 776-780 (2007).
31. Avraham-Davidi, I., et al. ApoB-containing lipoproteins regulate angiogenesis by modulating expression of VEGF receptor 1. Nat Med 18, 967-973 (2012).
32. Kim, S., et al. Carcinoma-produced factors activate myeloid cells through TLR2 to stimulate metastasis. Nature 457, 102-106 (2009).
33. Malanchi, I., et al. Interactions between cancer stem cells and their niche govern metastatic colonization. Nature 481, 85-89 (2012).
34. Oskarsson, T., et al. Breast cancer cells produce tenascin C as a metastatic niche component to colonize the lungs. Nat Med 17, 867-874 (2011).
35. Soikkeli, J., et al. Metastatic outgrowth encompasses COL-I, FN1, and POSTN up-regulation and assembly to fibrillar networks regulating cell adhesion, migration, and growth. Am J Pathol 177, 387-403 (2010).
36. Bierie, B. & Moses, H. L. Tumour microenvironment: TGFbeta: the molecular Jekyll and Hyde of cancer. Nat Rev Cancer 6, 506-520 (2006).
37. Matsumoto, K., Yoshitomi, H., Rossant, J. & Zaret, K. S. Liver organogenesis promoted by endothelial cells prior to vascular function. Science 294, 559-563 (2001).
38. Ding, B. S., et al. Endothelial-derived angiocrine signals induce and sustain regenerative lung alveolarization. Cell 147, 539-553 (2011).
39. Lammert, E., Cleaver, O. & Melton, D. Induction of pancreatic differentiation by signals from blood vessels. Science 294, 564-567 (2001).
40. Dodge, A. B., Lu, X. & D'Amore, P. A. Density-dependent endothelial cell production of an inhibitor of smooth muscle cell growth. J Cell Biochem 53, 21-31 (1993).
41. Crisan, M., et al. A perivascular origin for mesenchymal stem cells in multiple human organs. Cell Stem Cell 3, 301-313 (2008).
42. Kobayashi, H., et al. Angiocrine factors from Akt-activated endothelial cells balance self-renewal and differentiation of haematopoietic stem cells. Nat Cell Biol 12, 1046-1056 (2010).
43. Shen, Q., et al. Endothelial cells stimulate self-renewal and expand neurogenesis of neural stem cells. Science 304, 1338-1340 (2004).
44. Ding, L., Saunders, T. L., Enikolopov, G. & Morrison, S. J. Endothelial and perivascular cells maintain haematopoietic stem cells. Nature 481, 457-462 (2012).
45. Bandyopadhyay, S., et al. Interaction of KAI1 on tumor cells with DARC on vascular endothelium leads to metastasis suppression. Nat Med 12, 933-938 (2006).
46. Calabrese, C., et al. A perivascular niche for brain tumor stem cells. Cancer Cell 11, 69-82 (2007).
47. Franses, J. W., Baker, A. B., Chitalia, V. C. & Edelman, E. R. Stromal endothelial cells directly influence cancer progression. Sci Transl Med 3, 66ra65 (2011).
48. Indraccolo, S., et al. Cross-talk between tumor and endothelial cells involving the Notch3-D114 interaction marks escape from tumor dormancy. Cancer Res 69, 1314-1323 (2009).
49. Panigrahy, D., et al. Epoxyeicosanoids stimulate multiorgan metastasis and tumor dormancy escape in mice. J Clin Invest 122, 178-191 (2012).
50. Asosingh, K., et al. Nascent endothelium initiates th2 polarization of asthma. J Immunol 190, 3458-3465 (2013).
51. Lin, E. Y., Nguyen, A. V., Russell, R. G. & Pollard, J. W. Colony-stimulating factor 1 promotes progression of mammary tumors to malignancy. J Exp Med 193, 727-740 (2001).
52. Qian, B. Z. & Pollard, J. W. Macrophage diversity enhances tumor progression and metastasis. Cell 141, 39-51 (2010).
53. DeNardo, D. G., et al. CD4(+) T cells regulate pulmonary metastasis of mammary carcinomas by enhancing protumor properties of macrophages. Cancer Cell 16, 91-102 (2009).
54. Folkman, J. Angiogenesis: an organizing principle for drug discovery? Nat Rev Drug Discov 6, 273-286 (2007).
55. Jakobsson, L., et al. Endothelial cells dynamically compete for the tip cell position during angiogenic sprouting. Nat Cell Biol 12, 943-953 (2010).
56. Brooks, P. C., Clark, R. A. & Cheresh, D. A. Requirement of vascular integrin alpha v beta 3 for angiogenesis. Science 264, 569-571 (1994).
57. Stratman, A. N., Davis, M. J. & Davis, G. E. VEGF and FGF prime vascular tube morphogenesis and sprouting directed by hematopoietic stem cell cytokines. Blood 117, 3709-3719 (2011).
58. Pitulescu, M. E., Schmidt, I., Benedito, R. & Adams, R. H. Inducible gene targeting in the neonatal vasculature and analysis of retinal angiogenesis in mice. Nat Protoc 5, 1518-1534 (2010).
59. Herbert, S. P., et al. Arterial-venous segregation by selective cell sprouting: an alternativemode of blood vessel formation. Science 326, 294-298 (2009).
60. Nicoli, S. & Presta, M. The zebrafish/tumor xenograft angiogenesis assay. Nat Protoc 2, 2918-2923 (2007).
61. Baluk, P., Morikawa, S., Haskell, A., Mancuso, M. & McDonald, D. M. Abnormalities of basement membrane on blood vessels and endothelial sprouts in tumors. Am J Pathol 163, 1801-1815 (2003).
62. Ghajar, C. M., et al. The effect of matrix density on the regulation of 3-D capillary morphogenesis. Biophys J 94, 1930-1941 (2008).
63. Tanner, K., Mori, H., Mroue, R., Bruni-Cardoso, A. & Bissell, M. J. Coherent angular motion in the establishment of multicellular architecture of glandular tissues. Proc Natl Acad Sci USA (2012).
64. Lee, G. Y., Kenny, P. A., Lee, E. H. & Bissell, M. J. Three-dimensional culture models of normal and malignant breast epithelial cells. Nat Methods 4, 359-365 (2007).
65. Beliveau, A., et al. Raf-induced MMP9 disrupts tissue architecture of human breast cells in three-dimensional culture and is necessary for tumor growth in vivo. Genes Dev 24, 2800-2811 (2010).

What is claimed is:

1. A tissue model for in vitro organotypic modeling of dormancy in a microvascular niche comprising: (a) stromal cells of a selected specific stromal cell type from a particular tissue; (b) endothelial cells, wherein the endothelial cells are from the particular tissue or human umbilical vein endothelial cells (HUVEC), wherein the stromal cells and the endothelial cells self-assembled to form a microvascular niche, and (c) seeded cells of interest.

2. The tissue model of claim 1 further comprising other seeded resident cells, wherein the resident cells are cells that reside in vivo in the particular tissue being modeled.

3. The tissue model of claim 1 further comprising seeded non-resident cells, wherein the non-resident cells are cells that do not reside in or are generated in vivo from the particular tissue being modeled.

4. The tissue model of claim 1, wherein the tissue is lung, brain, bone marrow, liver, lymph node, ovary, omentum, pancreas, skeletal muscle, heart, skin, bladder, breast, prostate, kidney, or bladder.

5. A method for forming a synthetic organotypic model of dormancy in a microvascular niche in a tissue comprising the steps of (a) contacting stromal cells with endothelial cells, wherein said stromal cells are of a specific cell type from the tissue being modeled, (b) forming three-dimensional (3D) complexes through self-assembly of the stromal cells and endothelial cells to simulate native microvascular niches; and (c) culturing or seeding cells of interest in the 3D complexes.

6. The method of claim 5, further comprising the step of (d) detecting dormancy or growth of said seeded cells.

7. The method of claim 5, wherein said endothelial cells are human umbilical vein endothelial cells (HUVEC).

8. The method of claim 7, wherein said HUVEC are transduced with a lentiviral construct containing the human adenoviral E4ORF1 gene.

9. The method of claim 5, wherein said endothelial cells are resident endothelial cells from the particular tissue being modeled, wherein the tissue being modeled is lung, bone marrow, liver, brain, lymph node, ovary, omentum, pancreas, skeletal muscle, heart, skin, bladder, breast, prostate, kidney, or bladder.

10. The method of claim 5, wherein the tissue being modeled is lung, bone marrow, liver, brain, or breast.

11. A lung tissue microvascular niche model formed by the method of claim 10, wherein said model comprising lung fibroblasts, and human umbilical vein endothelial cells (HUVEC) or lung endothelial cells.

12. A bone marrow microvascular niche formed by the method of claim 10, wherein said model comprising mesenchymal stem cells, and human umbilical vein endothelial cells (HUVEC) or bone marrow endothelial cells.

13. A brain microvascular niche formed by the method of claim 10, wherein said model comprising human adventitial fibroblasts and astrocytes, and human umbilical vein endothelial cells (HUVEC) or endothelial cells.

14. A liver microvascular niche formed by the method of claim 10, wherein said model comprising liver fibroblasts, and endothelial cells or human umbilical vein endothelial cells (HUVEC).

15. A method for screening comprising the steps of: (a) forming a synthetic organotypic model of dormancy in a tissue microvascular niche model, wherein the forming steps comprising the steps of the method of claim 5; (b) adding patient-derived tumor cells to the formed microvasculature niche model; (c) allowing the tumor cells to become dormant; and (d) screening for molecules of interest that have therapeutic efficacy against dormant tumor cells.

16. The method of claim 15, wherein the molecules of interest are small molecules, peptides, antibodies, siRNAs, or antisense molecules.

17. The method of claim 15, wherein the molecules of interest are molecules that sensitize the dormant tumor cells to chemotherapeutic agents, radiation, targeted agents, or any combination thereof.

18. A method for screening comprising the steps of: (a) forming a microvascular niche modelin a tissue wherein said forming step comprising the steps of (i) contacting stromal cells with endothelial cells, wherein said stromal cells are of a specific cell type from the tissue being modeled, (ii) forming three-dimensional (3D) complexes through self-assembly of the stromal cells and endothelial cells to simulate native microvascular niches; and (iii) culturing or seeding cells of interest in the 3D complexes, wherein the seeded cells of interest are localized tumor cells from a patient that are seeded onto the formed microvasculature niche model; and (b) determining at various time points if any growth of the tumor cells occurs to assess the capacity of a patient's tumor for dormancy or metastatic colonization.

19. The method of claim 18, further comprising step (c) contacting a drug or therapeutic with said cells in said microvasculature niche model to assess the efficacy of a particular drug or therapeutic compound against a patient's tumor cells.

20. A method for screening comprising: (a) forming a microvascular niche model seeded with cells of interest; (b) administering compounds to the seeded cells; (c) profiling the RNA or protein levels of the cells of interest grown in the microvascular niche; (d) comparing the RNA or protein profiles between microvascular niche versus stroma alone; and (e) identifying compounds that drive tumor cells into a dormant state.

21. A method for screening comprising the steps of: (a) forming microvascular niche models with different densities of neovascular tips seeded with cells, (b) administering molecules of interest to the seeded cells; (c) profiling the RNA or protein levels of the cells of interest grown in the microvascular niche; (d) comparing the RNA or protein profiles between microvascular niches with different tip densities; and (e) identifying molecules of interest with pro-metastatic functions.
